SEQUENCE LISTING (8 sequences)

SEQ ID NO: 1 - protein, 5 aa, synthetic construct
  REGION 1..5: note = Synthetic peptide
VYYMN

SEQ ID NO: 2 - protein, 17 aa, synthetic construct
  REGION 1..17: note = Synthetic peptide
DINNEGGTTYYADSVKG

SEQ ID NO: 3 - protein, 13 aa, synthetic construct
  REGION 1..13: note = Synthetic peptide
DAGYSNHVPIFDS

SEQ ID NO: 4 - protein, 14 aa, synthetic construct
  REGION 1..14: note = Synthetic peptide
GLKSGSVTSDNFPT

SEQ ID NO: 5 - protein, 7 aa, synthetic construct
  REGION 1..7: note = Synthetic peptide
NTNTRHS

SEQ ID NO: 6 - protein, 10 aa, synthetic construct
  REGION 1..10: note = Synthetic peptide
ALFISNPSVE

SEQ ID NO: 7 - protein, 122 aa, synthetic construct
  REGION 1..122: note = Synthetic polypeptide
EVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSS

SEQ ID NO: 8 - protein, 111 aa, synthetic construct
  REGION 1..111: note = Synthetic polypeptide
QAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLG

(19) United States
(12) Patent Application Publication
(10) Pub. No.: US 2024/0182590 A1
(43) Pub. Date: Jun. 6, 2024

(54) METHOD OF TREATMENT OF PATIENTS HAVING REDUCED SENSITIVITY TO A BCL-2 INHIBITOR

(71) Applicants: argenx BV, Ghent (BE); The Regents of the University of Colorado, A Body Corporate, Denver, CO (US)

(72) Inventors: Johannes Joseph Wilhelmus DE HAARD, Ghent (BE); Anna HULTBERG, Hull, MA (US); Julie JACOBS, Weelde (BE); Piotr ZABROCKI, Ghent (BE); Clayton SMITH, Aurora, CO (US); Craig JORDAN, Aurora, CO (US)

(21) Appl. No.: 18/175,225

(22) Filed: Feb. 27, 2023

Related U.S. Application Data
(63) Continuation of application No. PCT/EP21/73816, filed on Aug. 27, 2021.
(60) Provisional application No. 63/072,113, filed on Aug. 29, 2020.

(51) Int. Cl. C07K 16/28; A61K 31/496; A61K 31/708; A61K 39/00; A61P 35/02; G01N 33/50

(52) U.S. Cl. CPC C07K 16/2875; A61K 31/496; A61K 31/708; A61P 35/02; G01N 33/5044; A61K 2039/505; G01N 2800/7028

(57) ABSTRACT

Provided are an antibody or antigen binding fragment thereof that binds to CD70 for use in treating a myeloid malignancy in a human subject, who is resistant to BCL-2 inhibitor treatment and methods of treating a myeloid malignancy in a subject, said method comprising (a) selecting a human subject having a myeloid malignancy that has a reduced sensitivity or is refractory to a BCL-2 inhibitor; and (b) administering to the subject an antibody or antigen binding fragment thereof that binds to CD70. In certain embodiments, the myeloid malignancy is acute myeloid leukemia (AML). In certain embodiments, the antibody that binds to CD70 is cusatuzumab. In certain embodiments, a BCL-2 inhibitor is co-administered with the antibody or antigen binding fragment thereof that binds to CD70. In certain embodiments, the BCL-2 inhibitor is venetoclax.

### CROSS-REFERENCE TO RELATED APPLICATIONS

This Application is a Continuation of International Application PCT/EP2021/073816, filed Aug. 27, 2021, which claims priority to U.S. Provisional Application No. 63/072,113, filed Aug. 29, 2020. The entire disclosures of each of the aforementioned applications are incorporated herein by reference in their entirety.

### SEQUENCE LISTING

The instant application contains a Sequence Listing which has been filed electronically in ASCII ST.26 format and is hereby incorporated by reference in its entirety. Said ASCII ST.26 copy, created on Feb. 27, 2023, is named 195408_SL and is 11,270 bytes in size.

### FIELD OF INVENTION

The present invention relates to therapies, including combination therapies, for the treatment of cancer, particularly relapsed or refractory myeloid malignancy. The therapies are particularly useful for the treatment of acute myeloid leukemia (AML), including monocytic AML. The combination therapies include an antibody or antigen binding fragment thereof that binds to CD70 and a BCL-2 inhibitor, for example venetoclax or a pharmaceutically acceptable salt thereof.

### BACKGROUND OF INVENTION

In recent years, the development of new cancer treatments has focused on molecular targets, particularly proteins, implicated in cancer progression. The list of molecular targets involved in tumor growth, invasion and metastasis continues to expand, and includes proteins overexpressed by tumor cells as well as targets associated with systems supporting tumor growth such as the vasculature and immune system. The number of therapeutic or anti-cancer agents designed to interact with these molecular targets also continues to increase. A large number of targeted cancer medicines are now approved for clinical use, with many more in the developmental pipeline.

CD70 has been identified as a molecular target of particular interest owing to its constitutive expression on many types of hematological malignancies and solid carcinomas. Junker et al. (2005) J Urol. 173: 2150-3; Sloan et al. (2004) Am J Pathol. 164: 315-23; Held-Feindt and Mentlein (2002) Int J Cancer 98: 352-6; Hishima et al. (2000) Am J Surg Pathol. 24: 742-6; Lens et al. (1999) Br J Haematol. 106: 491-503; Boursalian et al. (2009) Adv Exp Med Biol. 647: 108-119; Wajant H. (2016) Expert Opin Ther Targets 20(8): 959-973. CD70 is a type II transmembrane glycoprotein belonging to the tumor necrosis factor (TNF) superfamily, which mediates its effects through binding to its cognate cell surface receptor, CD27. Both CD70 and CD27 are expressed by multiple cell types of the immune system, and the CD70-CD27 signaling pathway has been implicated in the regulation of several different aspects of the immune response. This is reflected in the fact that CD70 overexpression occurs in various autoimmune diseases including rheumatoid and psoriatic arthritis and lupus. Boursalian et al. (2009) Adv Exp Med Biol. 647: 108-119; Han et al. (2005) Lupus 14(8): 598-606; Lee et al. (2007) J Immunol. 179(4): 2609-2615; Oelke et al. (2004) Arthritis Rheum. 50(6): 1850-1860.

CD70 expression has been linked to poor prognosis for several cancers including B cell lymphoma, renal cell carcinoma and breast cancer. Bertrand et al. (2013) Genes Chromosomes Cancer 52(8): 764-774; Jilaveanu et al. (2012) Hum Pathol. 43(9): 1394-1399; Petrau et al. (2014) J Cancer 5(9): 761-764. CD70 expression has also been found on metastatic tissue in a high percentage of cases, indicating a key role for this molecule in cancer progression. Jacobs et al. (2015) Oncotarget 6(15): 13462-13475. Constitutive expression of CD70 and its receptor CD27 on tumor cells of hematopoietic lineage has been linked to a role of the CD70-CD27 signaling axis in directly regulating tumor cell proliferation and survival. Goto et al. (2012) Leuk Lymphoma 53(8): 1494-1500; Lens et al. (1999) Br J Haematol. 106(2); 491-503; Nilsson et al. (2005) Exp Hematol. 33(12): 1500-1507; van Doorn et al (2004) Cancer Res. 64(16): 5578-5586.

Upregulated CD70 expression on tumors, particularly solid tumors that do not co-express CD27, also contributes to immunosuppression in the tumor microenvironment in a variety of ways. For example, CD70 binding to CD27 on regulatory T cells (Tregs) has been shown to augment the frequency of Tregs, reduce tumor-specific T-cell responses, and promote tumor growth in mice. Claus et al. (2012) Cancer Res. 72(14): 3664-3676. CD70-CD27 signaling can also dampen the immune response by tumor-induced apoptosis of T lymphocytes, as demonstrated in renal cell carcinoma, glioma, and glioblastoma cells. Chahlavi et al. (2005) Cancer Res. 65(12): 5428-5438; Diegmann et al. (2006) Neoplasia 8(11): 933-938); Wischusen et al. (2002) Cancer Res 62(9): 2592-2599. Finally, CD70 expression has also been linked to T cell exhaustion, whereby lymphocytes adopt a more differentiated phenotype and fail to kill tumor cells. Wang et al. (2012) Cancer Res 72(23): 6119-6129; Yang et al. (2014) Leukemia 28(9): 1872-1884.

BCL-2 (B-cell lymphoma 2) is an integral outer mitochondrial membrane protein that blocks the apoptotic death of some cells such as lymphocytes. Overexpression of BCL-2 in cancer cells confers resistance to apoptosis, and therefore inhibition of this protein can promote tumor cell death. Recent clinical trials have reported that addition of the highly specific BCL-2 inhibitor venetoclax to current standard-of-care therapy for acute myeloid leukemia (AML), e.g., hypomethylating agents (HMA), can greatly increase response rates and overall survival for newly diagnosed patients with AML (75 years or older or with comorbidities) that preclude them from intensive induction chemotherapy (Dinardo (2020), New England Journal of Medicine). These findings led to the recent FDA approval of this regimen for this population, and it is now considered to be the standard of care.

The combination of venetoclax and the HMA azacitidine results in a remission rate of approximately 70% in AML. However, a significant minority of patients do not achieve a remission and are refractory. In addition, the majority of patients who do achieve a remission ultimately relapse.

Therefore, a need still exists for improved therapies for the treatment of cancer, including CD70-expressing cancers such as myeloid malignancies.

### SUMMARY OF INVENTION

In a first aspect, the invention provides an anti-CD70 antibody or CD70-binding fragment thereof for use in treating a myeloid malignancy in a human subject who is resistant to BCL-2 inhibitor treatment.

A further aspect of the invention is a method of treating a myeloid malignancy in a human subject. The method includes the steps of:
(a) selecting a human subject having a myeloid malignancy that has a reduced sensitivity or is refractory to a BCL-2 inhibitor; and
(b) administering to the human subject an antibody or antigen binding fragment thereof that binds to CD70.

In certain embodiments, the BCL-2 inhibitor is venetoclax or a pharmaceutically acceptable salt thereof.

In certain embodiments, the myeloid malignancy is selected from the group consisting of acute myeloid leukemia (AML), myelodysplastic syndromes (MDS), myeloproliferative neoplasms (MPN), chronic myeloid leukemia (CML), and myelomonocytic leukemia (CMML).

In certain embodiments, the myeloid malignancy is AML.

In certain embodiments, the AML is monocytic AML.

In certain embodiments, the myeloid malignancy is MDS.

In certain embodiments, step (a) comprises determining an expression level of at least one marker selected from the group consisting of: BCL-2, CD117, CD11b, CD68, CD64, BCL2A1, and MCL1, of malignant myeloid cells of the human subject.

In certain embodiments, at least one of BCL-2 and CD117 is downregulated, and at least one of CD11b, CD68, CD64, CD70, BCL2A1, and MCL1 is upregulated.

In certain embodiments, step (a) comprises determining a CD70 expression level of malignant myeloid cells of the human subject.

In certain embodiments, CD70 is upregulated compared to a CD70 expression level as measured before or during a BCL-2 inhibitor treatment.

In certain embodiments, the human subject has a clinical history comprising:
(a) treatment with a BCL-2 inhibitor; and
(b) absence of a remission in response to the treatment with the BCL-2 inhibitor.

In certain embodiments, the historical treatment with the BCL-2 inhibitor further comprises treatment with a hypomethylating agent (HMA).

In certain embodiments, the human subject has a clinical history comprising:
(a) treatment with a BCL-2 inhibitor;
(b) partial or complete remission; and
(c) partial or complete relapse.

In certain embodiments, the historical treatment with the BCL-2 inhibitor further comprises treatment with a hypomethylating agent (HMA).

In certain embodiments, a hypomethylating agent (HMA) is co-administered with the antibody or antigen binding fragment thereof that binds to CD70.

In certain embodiments, a BCL-2 inhibitor is co-administered with the antibody or antigen binding fragment thereof that binds to CD70.

In certain embodiments, the antibody or antibody binding fragment that binds to CD70 comprises HCDR1, HCDR2, HCDR3, LCDR1, LCDR2, and LCDR3, wherein
the amino acid sequence of HCDR 1 consists of SEQ ID NO: 1;
the amino acid sequence of HCDR2 consists of SEQ ID NO: 2;
the amino acid sequence of HCDR3 consists of SEQ ID NO: 3;
the amino acid sequence of LCDR1 consists of SEQ ID NO: 4;
the amino acid sequence of LCDR2 consists of SEQ ID NO: 5; and
the amino acid sequence of LCDR3 consists of SEQ ID NO: 6.

In certain embodiments, the antibody or antibody binding fragment that binds to CD70 comprises a variable heavy chain domain (VH) comprising an amino acid sequence at least 90% identical to SEQ ID NO: 7 and a variable light chain domain (VL) comprising an amino acid sequence at least 90% identical to SEQ ID NO: 8.

In certain embodiments, the antibody or antibody binding fragment that binds to CD70 comprises a variable heavy chain domain (VH) comprising an amino acid sequence identical to SEQ ID NO: 7 and a variable light chain domain (VL) comprising an amino acid sequence identical to SEQ ID NO: 8.

In certain embodiments, the amino acid sequence which is at least 90% identical to the VH consisting of SEQ ID NO: 7 comprises HCDR1, HCDR2, and HCDR3, wherein
the amino acid sequence of HCDR1 consists of SEQ ID NO: 1;
the amino acid sequence of HCDR2 consists of SEQ ID NO: 2; and
the amino acid sequence of HCDR3 consists of SEQ ID NO: 3; and
wherein the amino acid sequence which is at least 90% identical to the VL consisting of SEQ ID NO: 8 comprises LCDR1, LCDR2, and LCDR3, wherein
the amino acid sequence of LCDR1 consists of SEQ ID NO: 4;
the amino acid sequence of LCDR2 consists of SEQ ID NO: 5; and
the amino acid sequence of LCDR3 consists of SEQ ID NO: 6.

In certain embodiments, the HMA is selected from the group consisting of azacitidine, decitabine, and guadecitabine.

In certain embodiments, the BCL-2 inhibitor is venetoclax or a pharmaceutically acceptable salt thereof.

In certain embodiments, the antibody that binds to CD70 is cusatuzumab.

An aspect of the invention is a method of identifying and treating a patient to be treated with an anti-CD70 antibody or antigen-binding fragment thereof, wherein the patient has a myeloid malignancy, the method comprising the steps of:
(i) measuring the myeloid differentiation status of the patient;
(ii) determining whether the patient has differentiated monocytic AML, wherein a patient having differentiated monocytic AML is identified as a patient to be treated with the anti-CD70 antibody or CD70-binding fragment thereof; and
(iii) administering the anti-CD70 antibody or CD70-binding fragment thereof to the patient identified as a patient to be treated with the anti-CD70 antibody or CD70-binding fragment thereof.

An aspect of the invention is an anti-CD70 antibody or CD70-binding fragment thereof for use in treating a myeloid malignancy in a patient who is resistant to BCL-2 inhibitor treatment.

A further aspect of the invention is an antibody or antigen binding fragment thereof that binds to CD70 for use in treating a myeloid malignancy in a patient who is resistant to BCL-2 inhibitor treatment.

In certain embodiments, the patient has received prior treatment with a BCL-2 inhibitor or with a BCL-2 inhibitor plus a hypomethylating agent (HMA).

In certain embodiments, the myeloid malignancy is selected from: acute myeloid leukemia (AML); myelodysplastic syndromes (MDS); myeloproliferative neoplasms (MPN); chronic myeloid leukemia (CML); and myelomonocytic leukemia (CMML).

In certain embodiments, the myeloid malignancy is AML or MDS.

In certain embodiments, the patient is identified on the basis of different expression levels as having differentiated monocytic AML.

In certain embodiments, the treatment is preceded by a selection comprising the steps of:
(i) measuring the myeloid differentiation status of the patient, and
(ii) determining whether the patient has differentiated monocytic AML, and
wherein a therapeutically effective dose of the anti-CD70 antibody or anti-CD70-binding fragment thereof is administered to said patient having differentiated monocytic AML.

In certain embodiments, the patient is identified as having differentiated monocytic AML on the basis of differential expression level(s) of at least one of the monocytic markers selected from the group consisting of: BCL-2, CD117, CD11b, CD68, CD64, BCL2A1, and MCL1.

In certain embodiments, the patient exhibits down-regulated expression of at least one of BCL-2 and CD117, and upregulated expression of at least one of CD11b, CD68, CD64, BCL2A1, and MCL1.

In certain embodiments, the human subject has a clinical history comprising:
(a) treatment with a BCL-2 inhibitor; and
(b) absence of a remission in response to the treatment with the BCL-2 inhibitor.

In certain embodiments, the historical treatment with the BCL-2 inhibitor further comprises treatment with a hypomethylating agent (HMA).

In certain embodiments, the human subject has a clinical history comprising:
(a) treatment with a BCL-2 inhibitor;
(b) partial or complete remission; and
(c) partial or complete relapse.

In certain embodiments, the historical treatment with the BCL-2 inhibitor further comprises treatment with a hypomethylating agent (HMA).

In certain embodiments, a hypomethylating agent (HMA) is co-administered with the antibody or antigen binding fragment thereof that binds to CD70.

In certain embodiments, a BCL-2 inhibitor is co-administered with the antibody or antigen binding fragment thereof that binds to CD70.

In certain embodiments, a BCL-2 inhibitor and a hypomethylating agent (HMA) is co-administered with the antibody or antigen binding fragment thereof that binds to CD70.

In certain embodiments, CD70 expression level in the patient is measured.

In certain embodiments, CD70 is upregulated compared to a CD70 expression level as measured before or during a BCL-2 inhibitor treatment.

In certain embodiments, the BCL-2 inhibitor resistant patient is a relapsed or refractory patient to a BCL-2 inhibitor.

In certain embodiments, the BCL-2 inhibitor is venetoclax or a pharmaceutically acceptable salt thereof.

In certain embodiments, the hypomethylating agent (HMA) is azacitidine, decitabine or guadecitabine.

In certain embodiments, the patient is resistant to a combination treatment with a BCL-2 inhibitor plus an HMA.

In certain embodiments, the patient is resistant to venetoclax plus azacitidine combination treatment.

In certain embodiments, the anti-CD70 antibody or CD70-binding fragment thereof comprises a variable heavy chain domain (VH) and a variable light chain domain (VL) wherein the VH and VL domains comprise the CDR sequences:
HCDR1 consisting of SEQ ID NO: 1;
HCDR2 consisting of SEQ ID NO: 2;
HCDR3 consisting of SEQ ID NO: 3;
LCDR1 consisting of SEQ ID NO: 4;
LCDR2 consisting of SEQ ID NO: 5; and
LCDR3 consisting of SEQ ID NO: 6.

In certain embodiments, the anti-CD70 antibody or anti-CD70-binding fragment thereof comprises a variable heavy chain domain (VH) consisting of SEQ ID NO: 7 or at least 90% identical thereto and a variable light chain domain (VL) consisting of SEQ ID NO: 8 or at least 90% identical thereto.

In certain embodiments, the amino acid difference in the amino acid sequence which is at least 90% identical to the VH consisting of SEQ ID NO: 7, is not in the CDR sequences of the VH; and the amino acid difference in the amino acid sequence which is at least 90% identical to the VL consisting of SEQ ID NO: 8, is not in the CDR sequences of the VL.

In certain embodiments, the anti-CD70 antibody is cusatuzumab.

In certain embodiments, a hypomethylating agent (HMA) is co-administered with the anti-CD70 antibody or anti-CD70-binding fragment thereof.

In certain embodiments, a BCL-2 inhibitor is co-administered with the anti-CD70 antibody or CD70-binding fragment thereof.

An aspect of the invention is a method of identifying a patient to be treated with an anti-CD70 antibody or antigen-binding fragment thereof, wherein the patient has a myeloid malignancy and is selected according to a method comprising the steps of:
(i) measuring the myeloid differentiation status of the patient, and
(ii) determining whether the patient has differentiated monocytic AML,
   wherein a patient having differentiated monocytic AML is identified as a patient to be treated with the anti-CD70 antibody or CD70-binding fragment thereof.

In certain embodiments, steps (i) and (ii) are performed in a sample obtained from the patient with a myeloid malignancy.

In certain embodiments, a bone marrow sample of the patient comprises CD45^{bright}/SSC^{high}/CD38⁺/CD34⁻/CD33⁺/CD11b⁺/CD70⁺ phenotype cells or CD45^{bright}/SSC^{high}/CD34⁻/CD117⁻/CD11b⁺/CD68⁺/CD14⁺/CD64⁺ phenotype cells.

### BRIEF DESCRIPTION OF THE DRAWINGS

FIG. 1A depicts treatment history of patient Pt-51 and flow analysis of bone marrow (BM) specimens at diagnosis. In the CD45/SSC plots, Mono, Prim, and Lym gates indicate monocytic, primitive, and lymphocytic subpopulations, respectively. The CD34/CD117 and CD68/CD11b plots show immunophenotype of the gated primitive subpopulations. Arrows highlight populations of interest. FIG. 1 adapted from Pei et al. 2020.

FIG. 1B depicts treatment history of patient Pt-72 and flow analysis of bone marrow (BM) specimens at diagnosis. In the CD45/SSC plots, Mono, Prim, and Lym gates indicate monocytic, primitive, and lymphocytic subpopulations, respectively. The CD34/CD117 and CD68/CD11b plots show immunophenotype of the gated monocytic subpopulations. Arrows highlight populations of interest. FIG. 1 adapted from Pei et al. 2020.

FIG. 1C depicts violin plots showing median fluorescence intensity (MFI) of CD117, CD11b, CD68, and CD64 in mono-AML (N=5) and prim-AML (N=7) quantified by flow cytometry analysis. Each dot represents a unique AML. Mann-Whitney test was used to determine significance. FIG. 1 adapted from Pei et al. 2020.

FIG. 1D depicts Viability of sorted ROS-low LSCs from mono-AML (N=5) and prim-AML (N=7) after 24 hours in vitro treatment with VEN alone or in combination with a fixed dose of 1.5 μmol/L AZA. Mean±SD of technical triplicates. All viability data were normalized to untreated (UNT) controls. VEN, venetoclax. AZA, azacitidine. FIG. 1 adapted from Pei et al. (2020).

FIG. 2A is a bar graph showing expression of BCL2 in ROS-low prim-AML (N=7) and ROS-low mono-AML (N=5). Each dot represents a unique AML. Mean+SD. FIG. 2 adapted from Pei et al. (2020).

FIG. 2B is a bar graph showing expression of BCL2 in FAB-M0 (N=16), M1 (N=44), M2 (N=40), M0/1/2 (N=100), and M5 (N=21) subclasses of AMLs from the TCGA (The Cancer Genome Atlas) dataset. Each dot represents a unique AML. FIG. 2 adapted from Pei et al. (2020).

FIG. 2C is an image of Western blot results showing protein-level expression of BCL-2 in prim-AML (N=5) and mono-AML (N=4). Actin is used as loading control. FIG. 2 adapted from Pei et al. (2020).

FIG. 3A depicts treatment history of patient Pt-12 and flow analysis of their diagnosis (Dx) and relapse (RI) specimens. In the CD45/SSC plots, Mono, Prim and Lym gates identify monocytic, primitive, and lymphocytic populations, respectively. The CD34/CD117 and CD68/CD11b plots show immunophenotype of the gated primitive subpopulations(P-AML) and monocytic subpopulations (M-AML). Arrows highlight populations of interest in the CD45/SSC plots, in particular the monocytic subpopulation. FIG. 3 adapted from Pei et al. (2020).

FIG. 3B depicts treatment history of patient Pt-65 and flow analysis of their diagnosis (Dx) and relapse (RI) specimens. In the CD45/SSC plots, Mono, Prim and Lym gates identify monocytic, primitive, and lymphocytic populations, respectively. The CD34/CD117 and CD68/CD11b plots show immunophenotype of the gated primitive subpopulations (P-AML) and monocytic subpopulations (M-MAL). Arrows highlight populations of interest in the CD45/SSC plots, in particular the monocytic subpopulation. FIG. 3 adapted from Pei et al. (2020).

FIG. 4 is a bar graph showing CD70 mRNA expression levels in FAB-M0 (N=13), M1 (N=39), M2 (N=37), M3 (N=14), M4 (N=32) and M5 (N=18) subclasses of AMLs. Patients with the highest CD70 expression belong to M5 subtype containing over 80% of monocytic AML cells in the bone marrow.

FIG. 5A and FIG. 5B are a flow cytometry analyses of bone marrow samples from VEN+AZA refractory monocytic disease (FIG. 5A) and VEN+AZA refractory containing mixed phenotype with monocytic and primitive AML cells (FIG. 5B). Gating of monocytic cells by CD34, CD11b, CD14 and CD64 shows higher levels of CD70 expression on monocytic AML cells as opposed to primitive cells (FIG. 5A and 5B). Primitive cells also show CD70 expression (FIG. 5B).

FIG. 6A depicts the comparison of median fluorescence intensity (MFI) for CD70 on primitive and monocytic AML cells in a bar graph (left), and a paired expression analysis per sample showing a higher CD70 expression level on monocytic AML cells than on primitive AML cells present in the same patient sample (right).

FIG. 6B depicts the comparison of percentage of CD70 positive primitive and monocytic AML cells in a bar graph (left) and a paired analysis of CD70 positive malignant cells per sample showing a higher percentage of CD70 expressing cells in monocytic AML cell populations (right). CD70 expression levels on monocytic malignant AML cells are higher than on primitive AML cells.

FIG. 7A is a flow cytometry analysis of mixed phenotype and monocytic AML samples used to assess NK-dependent killing of Cusatuzumab. In both samples monocytic CD38⁺/CD33⁺/CD11b+subpopulation expressed high levels of CD70 on the plasma membrane.

FIG. 7B is a bar graph showing the effect on monocytic and primitive AML cells. following administration of cusatuzumab, 41D12 FcDead antibody and a vehicle control. Cusatuzumab is able to significantly mediate NK-dependent cell killing of VEN+AZA sensitive mixed phenotype AML with monocytic and primitive AML cells. One-way ANOVA test was used to determine significance. *p<0.05.

FIG. 7C is a bar graph showing the effect on monocytic AML cells following administration of cusatuzumab, 41D12 FcDead antibody and a vehicle control. Cusatuzumab is able to significantly mediate NK-dependent cell killing of VEN+AZA resistant monocytic AML cells. One-way ANOVA test was used to determine significance. ***p<0.001.

FIG. 8 is a bar graph showing the median CD70 expression from transcriptomic analysis of gene expression performed on primitive and monocytic ROS-low LSCs from AML samples from bone marrow. Unpaired Wilcoxon test was used to compare both LSC subpopulations. *p<0.05.

FIG. 9 is a bar graph showing the effect of antibody treatment on leukemic stem cells from CD70 positive VEN+AZA resistant monocytic AML bone marrow samples. Data is normalized to the no antibody control colony forming units (CFU) for isotype control, blocking anti-CD70 antibody 41D12 FcDead and cusatuzumab. VEN+AZA resistant monocytic AML bone marrow samples were incubated with NK cells (1:5 T:E ratio) in the presence of antibodies (10 μg/ml) and then cultured in CFU medium in order to determine if LSCs were also efficiently targeted by cusatuzumab-mediated NK-dependent ADCC. Only cusatuzumab but not the control or the blocking antibody was able to significantly reduce the number of LSCs that give a rise to colonies in the medium. The plot shows data from 3 independent experiments with 3 different AML bone marrow samples from VEN+AZA resistant monocytic AML. One-way ANOVA test was used to determine significance. ****p<0.0001.

FIG. 10 is a bar graph showing the efficacy of anti-CD70 antibody treatment in the presence of NK cells in a patient-derived xenograft mouse model. NSGS mice were engrafted with VEN+AZA resistant monocytic AML bone marrow sample. When engraftment level in bone marrow reached around 25% of leukemic cells, animals were treated 3 times every 3 days with cusatuzumab or VEN+AZA with or without a single infusion of 1.5×10⁶ NK cells. After 9 days animals were sacrificed, bone marrow from femur isolated and the number of malignant monocytic cells in bone marrow was determined. Animals treated with cusatuzumab in combination with NK cells, but not cusatuzumab alone, VEN+AZA or VEN+AZA with NK cells, showed significantly reduced levels of monocytic AML cells in mouse bone marrow. Mann-Whitney test was used to determine significance. *p<0.05, **p<0.01, ***p<0.001, ****p<0.0001.

### DETAILED DESCRIPTION

### A. Definitions

Unless defined otherwise, all technical and scientific terms used herein have the same meaning as is commonly understood by one skilled in the art in the technical field of the invention.

Acute myeloid leukemia—As used herein, “acute myeloid leukemia” or “AML” refers to hematopoietic neoplasms involving myeloid cells. AML is characterized by clonal proliferation of myeloid precursors with reduced differentiation capacity. AML patients exhibit an accumulation of blast cells in the bone marrow. “Blast cells”, or simply “blasts”, as used herein refers to clonal myeloid progenitor cells exhibiting disrupted differentiation potential. Blast cells typically also accumulate in the peripheral blood of AML patients. Typically AML is diagnosed if the patient exhibits 20% or more blast cells in the bone marrow or peripheral blood. As used herein, the terms “patient” and “human subject” are used interchangeably.

According to the World Health Organization (WHO) classification scheme, AML in general encompasses the following subtypes: AML with recurrent genetic abnormalities; AML with myelodysplasia-related changes; therapy-related myeloid neoplasms; myeloid sarcoma; myeloid proliferations related to Down syndrome; blastic plasmacytoid dendritic cell neoplasm; and AML not otherwise categorized (e.g. acute megakaryoblastic leukemia, acute basophilic leukemia).

AML can also be categorized according to the French-American-British (FAB) classification system, encompassing the subtypes: MO (acute myeloblastic leukemia, minimally differentiated); M1 (acute myeloblastic leukemia, without maturation); M2 (acute myeloblastic leukemia, with granulocytic maturation); M3 (promyelocytic, or acute promyelocytic leukemia (APL)); M4 (acute myelomonocytic leukemia); M4eo (myelomonocytic together with bone marrow eosinophilia); M5 (acute monoblastic leukemia (M5a) or acute monocytic leukemia (M5b)); M6 (acute erythroid leukemias, including erythroleukemia (M6a) and very rare pure erythroid leukemia (M6b)); or M7 (acute megakaryoblastic leukemia).

Antibody—As used herein, the term “antibody” is intended to encompass full-length antibodies and variants thereof, including but not limited to modified antibodies, humanized antibodies, germlined antibodies. The term “antibody” is typically used herein to refer to immunoglobulin polypeptides having a combination of two heavy and two light chains wherein the polypeptide has significant specific immunoreactive activity to an antigen of interest (herein CD70). For antibodies of the IgG class, the antibodies comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region. The light chains of an antibody are classified as either kappa or lambda (κ,λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterized and are known to confer functional specialization. The term “antibody” as used herein encompasses antibodies from any class or subclass of antibody.

Antigen binding fragment—The term “antigen binding fragment” as used herein refers to fragments that are parts or portions of a full-length antibody or antibody chain comprising fewer amino acid residues than an intact or complete antibody whilst retaining antigen binding activity. An antigen-binding fragment of an antibody includes peptide fragments that exhibit specific immuno-reactive activity to the same antigen as the antibody (e.g., CD70). The term “antigen binding fragment” as used herein is intended to encompass antibody fragments selected from: an antibody light chain variable domain (VL); an antibody heavy chain variable domain (VH); a single chain antibody (scFv); a F(ab′)2 fragment; a Fab fragment; an Fd fragment; an Fv fragment; a one-armed (monovalent) antibody; diabodies, triabodies, tetrabodies or any antigen-binding molecule formed by combination, assembly or conjugation of such antigen binding fragments. The term “antigen binding fragment” as used herein may also encompass antibody fragments selected from the group consisting of: unibodies; domain antibodies; and nanobodies. Fragments can be obtained, for example, via chemical or enzymatic treatment of an intact or complete antibody or antibody chain or by recombinant means.

BCL-2—As used herein, “BCL-2” or the “BCL-2 protein” or “BCL2” refers to the first member of the BCL-2 protein family to be identified in humans, i.e., B-cell lymphoma 2. The cDNA encoding human BCL-2 was cloned in 1986 and the key role of this protein in inhibiting apoptosis was elucidated in 1988. BCL-2 has been found to be upregulated in several different types of cancer. For example, BCL-2 is activated by the t(14; 18) chromosomal translocation in follicular lymphoma. Amplification of the BOL-2 gene has also been reported in different cancers including leukemias (such as CLL), lymphomas (such as B-cell lymphoma) and some solid tumours (e.g. small-cell lung carcinoma). Human BCL-2 is encoded by the BCL2 gene (UniProtKB—P10415) and has the amino acid sequences shown under NCBI Reference Sequences NP_000624.2 and NP_000648.2.

BCL-2 family—As used herein, the term “BCL-2 family” or “BCL-2 protein family” refers to the collection of pro- and anti-apoptotic proteins related to BCL-2, see Delbridge et al. (2016) Nat Rev Cancer. 16(2): 99-109. There are at least 16 members of this family categorized into three functional groups: (i) the BCL-2 like proteins (e.g. BCL-2, BCL-X_{L/}BCL2L1, BCLW BCL2L2, MCL2, BFL1/BCL2A1); (ii) BAX and BAK; and (iii) the BH3-only proteins (e.g. BIM, PUMA, BAD, BMF, BID, NOXA, HRK, BIK). The BCL-2 family of proteins play an integral role in regulating the intrinsic apoptotic pathway with the anti-apoptotic members of the family (e.g. BCL-2, BCL-X_{L}) typically antagonizing the pro-apoptotic members (e.g. BAX and BIM). Deregulation of BCL-2 family members has been observed in many cancers, for example by gene translocations, amplifications, overexpression and mutations. The downstream effect of this deregulation is frequently apoptosis-resistance, which fuels cancer growth.

BCL2A1—As used herein, BCL2A1, or B-cell lymphoma 2-related protein Al, refers to an anti-apoptotic BCL2 protein that exerts important pro-survival functions. BCL2Al has been reported to be upregulated in AML and associated with resistance to venetoclax. Zhang et al. (2020) Nat Cancer 1: 826-839.

BCL-2 inhibitor—As used herein, a BCL-2 inhibitor refers to any agent, compound or molecule capable of specifically inhibiting the activity of BCL-2, in particular an agent, compound or molecule capable of inhibiting the anti-apoptotic activity of BCL-2. Examples of BCL-2 inhibitors suitable for use in the combinations described herein include B cell lymphoma homology 3 (BH3) mimetic compounds (Merino et al. (2018) Cancer Cell. 34(6): 879-891). Particular BCL-2 inhibitors include but are not limited to venetoclax, ABT-737 (Oltersdorf, T. et al. (2005) Nature 435: 677-681), navitoclax/ABT-263 (Tse, C. et al. (2008) Cancer Res. 68: 3421-3428), BM-1197 (Bai, L. et al. (2014) PLOS ONE 9: e99404), S44563 (Nemati, F. et al. (2014) PLOS ONE 9: e80836), BCL2-32 (Adam, A. et al. (2014) Blood 124: 5304), AZD4320 (Hennessy, E. J. et al. (2015) ACS Medicinal Chemistry annual meeting https://www.acsmedchem.org/ama/orig/abstracts/mediabstractf2015.pdf_abstr. 24), and S55746 (International Standard Randomised Controlled Trial Number Registry. ISRCTN http://www.isrctn.com/ISRCTN04804337 (2016). Further examples of BCL-2 inhibitors are described in Ashkenazi, A et al. (2017) Nature Reviews Drug Discovery 16: 273-284, incorporated herein by reference.

CD70—As used herein, the terms “CD70” or “CD70 protein” or “CD70 antigen” are used interchangeably and refer to a member of the TNF ligand family which is a ligand for TNFRSF7/CD27. CD70 is also known as CD27L or TNFSF7. The terms “human CD70” or “human CD70 protein” or “human CD70 antigen” are used interchangeably to refer specifically to the human homolog, including the native human CD70 protein naturally expressed in the human body and/or on the surface of cultured human cell lines, as well as recombinant forms and fragments thereof. Specific examples of human CD70 include the polypeptide having the amino acid sequence shown under NCBI Reference Sequence Accession No. NP_001243, or the extracellular domain thereof.

Cusatuzumab—As used herein “cusatuzumab,” also known as ARGX-110, is a monoclonal IgG1 anti-CD70 antibody. ARGX-110 has been shown to inhibit the interaction of CD70 with its receptor CD27 (Silence et al. (2014) MAbs. March-April;6(2): 523-32, incorporated herein by reference). In particular, ARGX-110 has been shown to inhibit CD70-induced CD27 signaling. Levels of CD27 signaling may be determined by, for example, measurement of serum soluble CD27 as described in Riether et al. (2017) J. Exp. Med. 214(2): 359-380) or of IL-8 expression as described in Silence et al. (2014) MAbs 6(2): 523-32. Without being bound by theory, inhibiting CD27 signaling is thought to reduce activation and/or proliferation of Treg cells, thereby reducing inhibition of anti-tumor effector T cells. ARGX-110 has also been demonstrated to deplete CD70-expressing tumor cells. In particular, ARGX-110 has been shown to lyse CD70-expressing tumor cells via antibody dependent cell-mediated cytotoxicity (ADCC) and complement dependent cytotoxicity (CDC), and also to increase antibody dependent cellular phagocytosis (ADCP) of CD70-expressing cells (Silence et al., Ibid.). ARGX-110 is afucosylated for enhanced ADCC.

The amino acid sequences of the six CDRs, VH, and VL of ARGX-110 or cusatuzumab are shown in Table 1.

TABLE 1

| | | SEQ |
|---|---|---|
| | | ID |
| ARGX-110 | Sequence | NO. |
| HCDR1 | VYYMN | 1 |
| HCDR2 | DINNEGGTTYYADSVKG | 2 |
| HCDR3 | DAGYSNHVPIFDS | 3 |
| LCDR1 | GLKSGSVTSDNFPT | 4 |
| LCDR2 | NTNTRHS | 5 |
| LCDR3 | ALFISNPSVE | 6 |
| VH | EVQLVESGGGLVQPGGSL | 7 |
| | RLSCAASGFTFSVYYMNW | |
| | VRQAPGKGLEWVSDINNE | |
| | GGTTYYADSVKGRFTISR | |
| | DNSKNSLYLQMNSLRAED | |
| | TAVYYCARDAGYSNHVPI | |
| | FDSWGQGTLVTVSS | |
| VL | QAVVTQEPSLTVSPGGTV | 8 |
| | TLTCGLKSGSVTSDNFPT | |
| | WYQQTPGQAPRLLIYNTN | |
| | TRHSGVPDRFSGSILGNK | |
| | AALTITGAQADDEAEYFC | |
| | ALFISNPSVEFGGGTQLT | |
| | VLG | |

Development stages of AML—Most cancers are staged based on the size and spread of tumors. The stages of AML are often characterized by blood cell counts and the accumulation of leukemia cells in other organs, like the liver or the spleen. The stage, or progression, of AML is an important factor in evaluating treatment options. Responses to a BCL-2 inhibitor (or a BCL-2 inhibitor plus a hypomethylating agent) in patients with AML correlate closely with developmental stage, where primitive AML is sensitive, but monocytic AML or “differentiated monocytic AML” (the terms are used interchangeably herein) is more resistant to a BCL-2 inhibitor therapy. Primary AML cells have different properties and thus exhibit different responses to therapies, from the more differentiated monocytic AML cells. Expression of monocytic markers may serve to distinguish between primary AML and monocytic AML cells. Such monocytic markers include BCL-2, CD117, CD11b, CD68, CD64, CD70, BCL2A1, MCL1, and other markers. Non-limiting examples of other monocytic markers include, CD38, CD34, CD33 and CD14. Monocytic AML cells may also be characterized as CD45^{bright} and SSC^{high} cells. Gene-expression levels of these monocytic markers are either more upregulated or downregulated on the monocytic tumor cells, depending on the development stage of AML. Myeloid differentiation status correlates with reduced BCL2 expression in patients with AML. Thus the more differentiated monocytic AML is much more likely to be refractory to BCL-2 inhibitor-based therapy.

Downregulated expression level—As used herein, “downregulated expression level” refers to a reduced expression level. This means a downward trend in the expression level of a monocytic marker. A downregulated expression level of a monocytic marker is a reduced expression level compared to an earlier expression level. An earlier expression level can be an expression level as measured in a patient before or during a BCL-2 inhibitor treatment (or before or during a treatment with a BCL-2 inhibitor and a hypomethylating agent). An earlier expression level can also be a baseline expression level of a monocytic marker on a monocytic tumor cell.

Historical treatment—As used herein, “historical treatment” refers to a previous treatment, e.g., an earlier treatment before a treatment with an antibody or antigen binding fragment thereof that binds to CD70.

Leukemic stem cells—As used herein, “leukemic stem cells” or “LSCs” are a subset of the blast cells associated with AML. LSCs are blast cells having stem cell properties such that, if transplanted into an immuno-deficient recipient, they are capable of initiating leukemic disease.

LSCs can self-renew by giving rise to leukemia and also partially differentiate into non-LSC conventional blast cells that resemble the original disease but are unable to self-renew. LSCs occur with a frequency in the range of 1 in 10,000 to 1 in 1 million as a proportion of primary AML blast cells (Pollyea and Jordan (2017) Blood 129: 1627-1635, incorporated herein by reference). LSCs may be characterized as cells that are CD34+, CD38−, optionally also CD45− and/or CD123+. LSCs may also be characterized as CD45dim, SSClow, CD90+CD34+ cells.

Myeloid malignancy—As used herein, the term “myeloid malignancy” refers to any clonal disease of hematopoietic stem or progenitor cells. Myeloid malignancies or myeloid malignant diseases include chronic and acute conditions. Chronic conditions include myelodysplastic syndromes (MDS), myeloproliferative neoplasms (MPN) and chronic myelomonocytic leukemia (CMML), and acute conditions include acute myeloid leukemia (AML).

NK-dependent ADCC—As used herein, “NK-dependent antibody-dependent cellular cytotoxicity (ADCC)” is an adaptive immune response mediated by natural killer (NK) cells. NK-dependent ADCC is initiated by activation of NK cells by antibodies. NK-dependent ADCC may be initiated by activation of NK cells by anti-CD70 antibodies.

Resistant—As used herein, the phrases “resistant to” or “resistance to” a therapy, for example resistance to a BCL-2 inhibitor therapy, refers to a reduced sensitivity to a treatment by a human subject. The term “resistant” includes upfront resistance to a therapy or relapse following initial response to a therapy. A patient may relapse, meaning that the patient initially responded to the therapy but ultimately relapsed; so the patient shows no positive response to a treatment anymore. The term “resistant” also includes, next to relapsed patients, refractory patients. A refractory response means that the patient shows no response at all to a given treatment. The patient does not achieve a remission and is refractory.

Standard intensive chemotherapy—As used herein, the phrase “standard intensive chemotherapy” (also referred to herein as “intensive induction therapy” or “induction therapy”) refers to the so-called “7+3” induction chemotherapy characterized by 7 days of high dose cytarabine followed by 3 days of anthracycline administration (e.g. daunorubicin or idarubicin). Standard intensive chemotherapy can be given to eligible newly-diagnosed AML patients with the aim of inducing complete remission of AML, typically with the intention of the patient undergoing a stem cell transplant following successful chemotherapy. As explained herein, not all newly-diagnosed AML patients are eligible for this standard intensive chemotherapy.

Upregulated expression level—As used herein, the phrase “upregulated expression level” refers to an elevated or higher expression level. This means an upward trend in the expression level of a monocytic marker. An upregulated expression level of a monocytic marker is a higher expression level compared to an earlier expression level. An earlier expression level can be an expression level as measured in a patient before or during a BCL-2 inhibitor treatment (or before or during a treatment with a BCL-2 inhibitor and a hypomethylating agent). An earlier expression level can also be a baseline expression level of a monocytic marker on a monocytic tumor cell.

Venetoclax—As used herein, the term “venetoclax” refers to the compound having the chemical structure shown below:

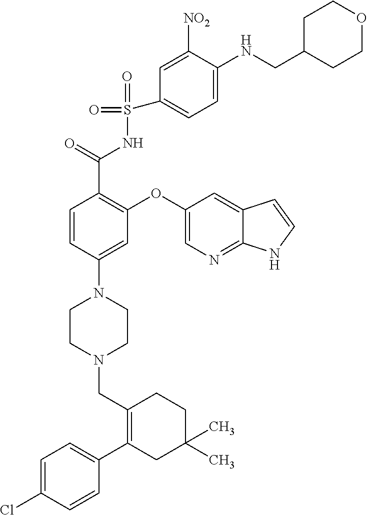 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax is a potent, selective, orally-bioavailable inhibitor of the BCL-2 protein. It has the empirical formula C₄₅H₅₀ClN₇O₇S and a molecular weight of 868.44. It has very low aqueous solubility. Venetoclax can be described chemically as 4-(4-{[2-(4-chlorophenyl)-4,4dimethylcyclohex-1-en-1-yl]methyl}piperazin-1-yl)-N-({3-nitro-4-[(tetrahydro-2H-pyran-4ylmethyl)amino]phenyl}sulfonyl)-2-(1H-pyrrolo[2,3-b]pyridin-5-yloxy)benzamide). Alternative names for venetoclax include ABT-199; chemical name 1257044-40-8; GDC-0199.

Venetoclax received approval from the US Food and Drug Administration (FDA) in 2015 for the treatment of adult patients with chronic lymphocytic leukemia (CLL) or small lymphocytic leukemia (SLL) who have received at least one prior therapy. Venetoclax is distributed and marketed by Abb Vie Inc. under the trade name VENCLEXTA®. Venetoclax is also approved in the US for use in combination with azacitidine or decitabine or low-dose cytarabine for the treatment of newly-diagnosed acute myeloid leukemia (AML) in adults aged 75 years or older or who have comorbidities that preclude use of intensive induction chemotherapy.

### B. Methods

The BCL-2 protein is an anti-apoptotic member of the BCL-2 family and is up-regulated in many different types of cancer. The overexpression of BCL-2 allows tumour cells to evade apoptosis by sequestering pro-apoptotic proteins. BCL-2 is highly expressed in many hematologic malignancies and is the predominant pro-survival protein in diseases such as chronic lymphocytic leukemia (CLL), follicular lymphoma and mantle cell lymphoma. Inhibition of BCL-2 inhibits the anti-apoptotic or pro-survival activity of this protein.

Anti-apoptotic members of the BCL-2 family, including BCL-2, have been reported as overexpressed in primary AML samples (Bogenberger et al. (2014) Leukemia 28(2): 1657-65). BCL-2 overexpression has also been reported in leukemic stem cells (LSCs) obtained from AML patients (Lagadinou et al. (2013) Cell Stem Cell 12(3): 329-341). Inhibition of BCL-2 in ex vivo LSC populations led to selective eradication of quiescent LSCs (Lagadinou et al. (2013) Cell Stem Cell 12(3): 329-341).

Without wishing to be bound by theory, the methods of the present invention are considered to be particularly effective for the treatment of AML due to the combined therapeutic effect of the CD70 antibodies or antigen binding fragments thereof and the BCL-2 inhibitor, particularly the combined effect at the level of the LSCs. The self-renewal capacity of LSCs means that the persistence of these cells is a major factor contributing to disease relapse.

In addition, the inventors have surprisingly found that the proportion of monocytic AML cells are increased in patients with myeloid malignancies that are refractory to treatment with the BCL-2 inhibitors venetoclax and hypermethylating agent (HMA) azacitidine. Consequently, it has been found that the presence of monocytic AML cells increases the risk of disease relapse.

The inventors have also determined that the monocytic AML cells express significantly higher CD70 levels relative to less differentiated primitive AML cells. Venetoclax and azacitidine resistant monocytic AML cells were sensitive to treatment with an anti-CD70 antibody both in vitro and in an in vivo mouse disease model. Without wishing to be bound by theory, the monocytic AML cells were considered to be targeted by an anti-CD70 antibody-mediated NK cell-dependent ADCC. Therefore, the anti-CD70 antibodies as described herein are considered especially effective for treating myeloid malignancies that are resistant to BCL-2 inhibitors such as venetoclax. In an aspect, the present invention provides a method for treating a myeloid malignancy in a human subject. The method is of particular use in the treatment of human subjects having a myeloid malignancy that has a reduced sensitivity or is refractory to a BCL-2 inhibitor such as venetoclax. The method includes the steps of
(a) selecting a human subject having a myeloid malignancy that has a reduced sensitivity or is refractory to a BCL-2 inhibitor; and
(b) administering to the human subject an antibody or antigen binding fragment thereof that binds to CD70.

In certain embodiments, the human subject has failed treatment of the myeloid malignancy with a BCL-2 inhibitor. In certain embodiments, the human subject had a clinical response to treatment of the myeloid malignancy with a BCL-2 inhibitor but subsequently suffered a relapse of the myeloid malignancy. The clinical response can be any clinical response, including a complete response, a partial response, or a minimal response. In certain embodiments, the human subject had a clinical response to treatment of the myeloid malignancy with a BCL-2 inhibitor but subsequently had a reduced clinical response to the BCL-2 inhibitor. In certain embodiments, the human subject had a clinical response to treatment of the myeloid malignancy with a BCL-2 inhibitor but subsequently became refractory to treatment with the BCL-2 inhibitor. In certain other embodiments, the human subject had no clinically significant response to treatment of the myeloid malignancy with a BCL-2 inhibitor.

In certain embodiments, the human subject has failed treatment of the myeloid malignancy with venetoclax or a pharmaceutically acceptable salt thereof. In certain embodiments, the human subject had a clinical response to treatment of the myeloid malignancy with venetoclax or a pharmaceutically acceptable salt thereof but subsequently suffered a relapse of the myeloid malignancy. The clinical response can be any clinical response, including a complete response, a partial response, or a minimal response. In certain embodiments, the human subject had a clinical response to treatment of the myeloid malignancy with venetoclax or a pharmaceutically acceptable salt thereof but subsequently had a reduced clinical response to venetoclax or a pharmaceutically acceptable salt thereof. In certain embodiments, the human subject had a clinical response to treatment of the myeloid malignancy with venetoclax or a pharmaceutically acceptable salt thereof but subsequently became refractory to treatment with venetoclax or a pharmaceutically acceptable salt thereof. In certain other embodiments, the human subject had no clinically significant response to treatment of the myeloid malignancy with venetoclax or a pharmaceutically acceptable salt thereof.

Venetoclax for use in the methods described herein may be provided in any suitable form such that it effectively inhibits the BCL-2 protein. Such forms include but are not limited to any suitable polymorphic, amorphous or crystalline forms or any isomeric or tautomeric forms. In certain embodiments, the combination therapies described herein comprise venetoclax synthesized according to the process described in US2010/0305122 (incorporated herein by reference). In alternative embodiments, the methods described herein comprise venetoclax according to the forms or synthesized according to the processes described in any one of CN107089981 (A), CN107648185 (A), EP3333167, WO2017/156398, WO2017/212431, WO2018/009444, WO2018/029711, WO2018/069941, WO2018/157803, and WO2018/167652 (each incorporated herein by reference). In certain embodiments, the methods described herein comprise venetoclax in any of the crystalline or salt forms described in WO2012/071336 (incorporated herein by reference).

Pharmaceutically acceptable salts for use in accordance with the present invention include salts of acidic or basic groups. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene -bis-(2-hydroxy-3-naphthoate)) salts.

Pharmaceutically acceptable salts may be formed with various amino acids. Suitable base salts include, but are not limited to, aluminum, calcium, lithium, magnesium, potassium, sodium, zinc, and diethanolamine salts.

In certain embodiments, the human subject has failed treatment of the myeloid malignancy with venetoclax. In certain embodiments, the human subject had a clinical response to treatment of the myeloid malignancy with venetoclax but subsequently suffered a relapse of the myeloid malignancy. The clinical response can be any clinical response, including a complete response, a partial response, or a minimal response. In certain embodiments, the human subject had a clinical response to treatment of the myeloid malignancy with venetoclax but subsequently had a reduced clinical response to venetoclax. In certain embodiments, the human subject had a clinical response to treatment of the myeloid malignancy with venetoclax but subsequently became refractory to treatment with venetoclax. In certain other embodiments, the human subject had no clinically significant response to treatment of the myeloid malignancy with venetoclax.

The method includes the step of administering to the human subject an antibody or antigen binding fragment thereof that binds to CD70. The administering can be achieved using any suitable route of administration, including, without limitation, oral, other parenteral, intravenous, intraperitoneal, pulmonary, and subcutaneous. For parenteral routes of administration (e.g., intravenous, intraperitoneal, pulmonary, and subcutaneous), the antibody or antigen binding fragment thereof that binds to CD70 can be administered to the human subject as an injection or as an infusion.

As described elsewhere herein, CD70 has already been characterized as an attractive target for anti-cancer therapy. CD70 is constitutively expressed on many types of hematological malignancies and solid carcinomas and its expression has been linked to poor prognosis for several cancers. Antibodies targeting CD70 have been developed and some have been taken forward into clinical development.

Antibodies targeting CD70 have been found to be particularly effective for the treatment of myeloid malignancies, particularly the treatment of subjects with acute myeloid leukemia (AML). The results from a Phase I/II clinical trial testing the CD70 antibody, ARGX-110 (cusatuzumab), in patients having AML revealed surprising efficacy in this indication, particularly in newly-diagnosed patients classified as unfit for standard intensive chemotherapy (see WO2018/229303). It is particularly notable that in the clinical studies, the CD70 antibody, when used in combination with azacitidine, efficiently reduced leukemic stem cells (LSCs) in the AML patients. Testing of the LSCs isolated from the patients in the trial revealed evidence of increased asymmetric division of LSCs, indicative of differentiation into myeloid cells. Taken together, these results indicate that CD70 antibodies deplete the LSC pool in AML patients thereby increasing the prospect of remission and reducing the risk of relapse.

In certain embodiments, the antibody that binds to CD70 is cusatuzumab.

Additional CD70 antibody or antigen binding fragments thereof that may be used in the methods described herein include antibody drug conjugates (ADCs). ADCs are antibodies attached to active agents, for example auristatins and maytansines or other cytotoxic agents. Certain ADCs maintain antibody blocking and/or effector function (e.g. ADCC, CDC, ADCP) while also delivering the conjugated active agent to cells expressing the target (e.g. CD70). Examples of anti-CD70 ADCs include vorsetuzumab mafodotin (also known as SGN-75, Seattle Genetics), SGN-70A (Seattle Genetics), and MDX-1203/BMS936561 (Bristol-Myers Squibb), each of which may be used in accordance with the invention. Suitable anti-CD70 ADCs are also described in WO2008074004 and WO2004073656, each of which is incorporated herein by reference.

In certain embodiments, the antigen binding fragment of the antibody that binds to CD70 is independently selected from the group consisting of: an antibody light chain variable domain (VL); an antibody heavy chain variable domain (VH); a single chain antibody (scFv); a F(ab′)2 fragment; a Fab fragment; an Fd fragment; an Fv fragment; a one-armed (monovalent) antibody; diabodies, triabodies, tetrabodies or any antigen-binding molecule formed by combination, assembly or conjugation of such antigen binding fragments.

In certain embodiments, the myeloid malignancy is selected from the group consisting of acute myeloid leukemia (AML), myelodysplastic syndromes (MDS), myeloproliferative neoplasms (MPN), chronic myeloid leukemia (CML), and chronic myelomonocytic leukemia (CMML). In certain embodiments, the myeloid malignancy is AML. In certain embodiments, the myeloid malignancy is MDS. In certain embodiments, the myeloid malignancy is MPN. In certain embodiments, the myeloid malignancy is CML. In certain embodiments, the myeloid malignancy is CMML.

As mentioned above, in certain embodiments, the myeloid malignancy is AML. Acute myeloid leukemia is also called acute myelocytic leukemia, acute myelogenous leukemia, acute granulocytic leukemia, and acute non-lymphocytic leukemia. The American Cancer Society's estimates for leukemia in the United States for 2020 include about 19,940 new cases of AML, mostly in adults; and about 11,180 deaths from AML, almost all in adults. AML is one of the most common types of leukemia in adults. Still, AML is fairly rare overall, accounting for only about 1% of all cancers. AML is generally a disease of older people and is uncommon before the age of 45. The average age of people when they are first diagnosed with AML is about 68, but AML can occur in children as well.

In certain embodiments, the myeloid malignancy is monocytic AML. Acute monocytic leukemia (AMOL, or AML-M5), also known as monoblastic AML, is considered a type of acute myeloid leukemia (AML). In order to fulfill World Health Organization (WHO) criteria for AML-M5, a patient must have greater than 20% blasts in the bone marrow, and of these, greater than 80% must be of the monocytic lineage.

As mentioned above, in certain embodiments, the myeloid malignancy is MDS. Myelodysplastic Syndromes (MDS) are a group of diverse bone marrow disorders (cancers) in which the bone marrow does not produce enough healthy blood cells. MDS is often referred to as a “bone marrow failure disorder.” In a patient with a myelodysplastic syndrome, the blood stem cells (immature cells) do not become mature red blood cells, white blood cells, or platelets in the bone marrow. These immature blood cells, called blasts, do not work the way they should and either die in the bone marrow or soon after they go into the blood. This leaves less room for healthy white blood cells, red blood cells, and platelets to form in the bone marrow. When there are fewer healthy blood cells, infection, anemia, or easy bleeding may occur. Various types of

MDS include, without limitation, refractory anemia, refractory anemia with ringed sideroblasts, refractory anemia with excess blasts, refractory cytopenia with multilineage dysplasia, refractory cytopenia with unilineage dysplasia, myelodysplastic syndrome associated with an isolated del(5q) chromosome abnormality, chronic myelomonocytic leukemia (CMML), and unclassifiable myelodysplastic syndrome.

In certain embodiments, step (a) comprises determining an expression level of at least one marker selected from the group consisting of: BCL-2, CD117, CD11b, CD68, CD64, BCL2A1, and MCL1, of malignant myeloid cells of the human subject. Relevant expression levels can be determined using any suitable method, including, without limitation, fluorescence-activated cell sorting (FACS), fluorescence microscopy using detectable (e.g., fluorescently labeled) antibodies specific for the relevant cell surface molecule(s) and mRNA expression analysis. In certain embodiments, step (a) comprises determining an expression level of at least one marker selected from the group consisting of: BCL-2, CD117, CD11b, CD68, CD64, BCL2A1, and MCL1, of malignant myeloid cells of the human subject. In certain embodiments, step (a) comprises determining an expression level of at least one marker selected from the group consisting of: BCL-2, CD117, CD11b, CD68, CD64, BCL2A1, and MCL1, of malignant myeloid cells of the human subject. In certain embodiments, step (a) comprises determining an expression level of BCL-2 of malignant myeloid cells of the human subject. In certain embodiments, step (a) comprises determining an expression level of CD117 of malignant myeloid cells of the human subject. In certain embodiments, step (a) comprises determining an expression level of CD11b of malignant myeloid cells of the human subject. In certain embodiments, step (a) comprises determining an expression level of CD68 of malignant myeloid cells of the human subject. In certain embodiments, step (a) comprises determining an expression level of CD64 of malignant myeloid cells of the human subject. In certain embodiments, step (a) comprises determining an expression level of BCL2A1 of malignant myeloid cells of the human subject. In certain embodiments, step (a) comprises determining an expression level of MCLI of malignant myeloid cells of the human subject.

In certain embodiments, at least one of BCL-2 and CD117 is downregulated, and at least one of CD11b, CD68, CD64, CD70, BCL2A1, and MCLI is upregulated. In certain embodiments, BCL-2 is downregulated and CD11b is upregulated. In certain embodiments, BCL-2 is downregulated and CD68 is upregulated. In certain embodiments, BCL-2 is downregulated and CD64 is upregulated. In certain embodiments, BCL-2 is downregulated and CD70 is upregulated. In certain embodiments, BCL-2 is downregulated and BCL2A1 is upregulated. In certain embodiments, BCL-2 is downregulated and MCL1 is upregulated. In certain embodiments, CD117 is downregulated and CD11b is upregulated. In certain embodiments, CD117 is downregulated and CD68 is upregulated. In certain embodiments, CD117 is downregulated and CD64 is upregulated. In certain embodiments, CD117 is downregulated and CD70 is upregulated. In certain embodiments, CD117 is downregulated and BCL2A1 is upregulated. In certain embodiments, CD117 is downregulated and MCL1 is upregulated.

In further embodiments, step (a) comprises determining an expression level of at least one marker selected from the group consisting of: CD117, CD11b and CD68. In certain embodiments, step (a) comprises determining an expression level of CD117 of malignant myeloid cells of the human subject. In certain embodiments, step (a) comprises determining an expression level of CD11b of malignant myeloid cells of the human subject. In certain embodiments, step (a) comprises determining an expression level of CD68 of malignant myeloid cells of the human subject. In certain embodiments, CD117 is downregulated. In certain embodiments, CD11b is upregulated. In certain embodiments, CD68 is upregulated. In certain embodiments, CD11b is upregulated and CD68 is upregulated. In certain embodiments, CD117 is downregulated, CD11b is upregulated and CD68 is upregulated.

In further embodiments, step (a) comprises determining an expression level of at least one marker selected from the group consisting of: CD64, CD34, CD117, CD11b, CD68 and CD14 of malignant myeloid cells of the human subject. In certain embodiments, step (a) comprises determining an expression level of CD64 of malignant myeloid cells of the human subject. In certain embodiments, step (a) comprises determining an expression level of CD34 of malignant myeloid cells of the human subject. In certain embodiments, step (a) comprises determining an expression level of CD117 of malignant myeloid cells of the human subject. In certain embodiments, step (a) comprises determining an expression level of CD11b of malignant myeloid cells of the human subject. In certain embodiments, step (a) comprises determining an expression level of CD68 of malignant myeloid cells of the human subject. In certain embodiments, step (a) comprises determining an expression level of CD14 of malignant myeloid cells of the human subject. In certain embodiments, CD64 is upregulated. In certain embodiments, CD34 is downregulated. In certain embodiments, CD117 is downregulated. In certain embodiments, CD11b is upregulated. In certain embodiments, CD68 is upregulated. In certain embodiments, CD14 is upregulated. In certain embodiments, CD64 is upregulated and CD34 is downregulated. In certain embodiments, CD64 is upregulated and CD117 is downregulated. In certain embodiments, CD64 is upregulated and CD11b is upregulated. In certain embodiments, CD64 is upregulated and CD68 is upregulated. In certain embodiments, CD64 is upregulated and CD14 is upregulated. In certain embodiments, CD34 is downregulated and CD117 is downregulated. In certain embodiments, CD34 is downregulated and CD11b is upregulated. In certain embodiments, CD34 is downregulated and CD68 is upregulated. In certain embodiments, CD34 is downregulated and CD14 is upregulated. In certain embodiments, CD117 is downregulated and CD14 is upregulated. In certain embodiments, CD68 is upregulated and CD14 is upregulated. In certain embodiments, CD64 is upregulated, CD34 is downregulated and CD117 is downregulated. In certain embodiments, CD64 is upregulated, CD34 is downregulated and CD11b is upregulated. In certain embodiments, CD64 is upregulated, CD34 is downregulated and CD68 is upregulated. In certain embodiments, CD64 is upregulated, CD34 is downregulated and CD14 is upregulated. In certain embodiments, CD64 is upregulated, CD117 is downregulated and CD14 is upregulated. In certain embodiments, CD64 is upregulated, CD68 is upregulated and CD14 is upregulated. In certain embodiments, CD34 is downregulated, CD117 is downregulated and CD11b is upregulated. In certain embodiments, CD34 is downregulated, CD117 is downregulated and CD68 is upregulated. In certain embodiments, CD34 is downregulated, CD11b is upregulated and CD68 is upregulated. In certain embodiments, CD34 is downregulated, CD117 is downregulated and CD14 is upregulated. In certain embodiments, CD34 is downregulated, CD11b is upregulated and CD14 is upregulated. In certain embodiments, CD117 is downregulated, CD11b is upregulated and CD14 is upregulated. In certain embodiments, CD34 is downregulated, CD68 is upregulated and CD14 is upregulated. In certain embodiments, CD117 is downregulated, CD68 is upregulated and CD14 is upregulated. In certain embodiments, CD11b is upregulated, CD68 is upregulated and CD14 is upregulated. In certain embodiments, CD64 is upregulated, CD34 is downregulated, CD117 is downregulated and CD11b is upregulated. In certain embodiments, CD64 is upregulated, CD34 is downregulated, CD117 is downregulated and CD68 is upregulated. In certain embodiments, CD64 is upregulated, CD34 is downregulated, CD11b is upregulated and CD68 is upregulated. In certain embodiments, CD64 is upregulated, CD34 is downregulated, CD117 is downregulated and CD14 is upregulated. In certain embodiments, CD64 is upregulated, CD34 is downregulated, CD11b is upregulated and CD14 is upregulated. In certain embodiments, CD64 is upregulated, CD117 is downregulated, CD11b is upregulated and CD14 is upregulated. In certain embodiments, CD64 is upregulated, CD34 is downregulated, CD68 is upregulated and CD14 is upregulated. In certain embodiments, CD64 is upregulated, CD117 is downregulated, CD68 is upregulated and CD14 is upregulated. In certain embodiments, CD64 is upregulated, CD11b is upregulated, CD68 is upregulated and CD14 is upregulated. In certain embodiments, CD34 is downregulated, CD117 is downregulated, CD11b is upregulated and CD68 is upregulated. In certain embodiments, CD34 is downregulated, CD117 is downregulated, CD11b is upregulated and CD14 is upregulated. In certain embodiments, CD34 is downregulated, CD117 is downregulated, CD68 is upregulated and CD14 is upregulated. In certain embodiments, CD34 is downregulated, CD11b is upregulated, CD68 is upregulated and CD14 is upregulated. In certain embodiments, CD117 is downregulated, CD11b is upregulated, CD68 is upregulated and CD14 is upregulated. In certain embodiments, CD64 is upregulated, CD34 is downregulated, CD117 is downregulated, CD11b is upregulated and CD68 is upregulated. In certain embodiments, CD64 is upregulated, CD34 is downregulated, CD117 is downregulated, CD11b is upregulated and CD14 is upregulated. In certain embodiments, CD64 is upregulated, CD34 is downregulated, CD117 is downregulated, CD68 is upregulated and CD14 is upregulated. In certain embodiments, CD64 is upregulated, CD34 is downregulated, CD11b is upregulated, CD68 is upregulated and CD14 is upregulated. In certain embodiments, CD64 is upregulated, CD117 is downregulated, CD11b is upregulated, CD68 is upregulated and CD14 is upregulated. In certain embodiments, CD34 is downregulated, CD117 is downregulated, CD11b is upregulated, CD68 is upregulated and CD14 is upregulated. In certain embodiments, CD64 is upregulated, CD34 is downregulated, CD117 is downregulated, CD11b is upregulated, CD68 is upregulated and CD14 is upregulated.

In further embodiments, step (a) comprises determining an expression level of at least one marker selected from the group consisting of: CD34, CD38, CD11b, CD33 and CD70 of malignant myeloid cells of the human subject. In certain embodiments, step (a) comprises determining an expression level of CD34 of malignant myeloid cells of the human subject. In certain embodiments, step (a) comprises determining an expression level of CD38 of malignant myeloid cells of the human subject. In certain embodiments, step (a) comprises determining an expression level of CD11b of malignant myeloid cells of the human subject. In certain embodiments, step (a) comprises determining an expression level of CD33 of malignant myeloid cells of the human subject. In certain embodiments, step (a) comprises determining an expression level of CD70 of malignant myeloid cells of the human subject. In certain embodiments, CD34 is downregulated. In certain embodiments, CD38 is upregulated. In certain embodiments, CD11b is upregulated. In certain embodiments, CD33 is upregulated. In certain embodiments, CD70 is upregulated. In certain embodiments, CD34 is downregulated and CD38 is upregulated. In certain embodiments, CD34 is downregulated and CD33 is upregulated. In certain embodiments, CD34 is downregulated and CD70 is upregulated. In certain embodiments, CD38 is upregulated and CD33 is upregulated. In certain embodiments, CD38 is upregulated and CD11b is upregulated. In certain embodiments, CD38 is upregulated and CD70 is upregulated. In certain embodiments, CD33 is upregulated and CD11b is upregulated. In certain embodiments, CD33 is upregulated and CD70 is upregulated. In certain embodiments, CD38 is upregulated, CD33 is upregulated and CD34 is downregulated. In certain embodiments, CD38 is upregulated, CD33 is upregulated and CD11b is upregulated. In certain embodiments, CD38 is upregulated, CD34 is downregulated and CD11b is upregulated. In certain embodiments, CD33 is upregulated, CD34 is downregulated and CD11b is upregulated. In certain embodiments, CD38 is upregulated, CD33 is upregulated and CD70 is upregulated. In certain embodiments, CD38 is upregulated, CD34 is downregulated and CD70 is upregulated. In certain embodiments, CD33 is upregulated, CD34 is downregulated and CD70 is upregulated. In certain embodiments, CD38 is upregulated, CD11b is upregulated and CD70 is upregulated. In certain embodiments, CD33 is upregulated, CD11b is upregulated and CD70 is upregulated. In certain embodiments, CD34 is downregulated, CD11b is upregulated and CD70 is upregulated. In certain embodiments, CD38 is upregulated, CD33 is upregulated, CD34 is downregulated and CD11b is upregulated. In certain embodiments, CD38 is upregulated, CD33 is upregulated, CD34 is downregulated and CD70 is upregulated. In certain embodiments, CD38 is upregulated, CD33 is upregulated, CD11b is upregulated and CD70 is upregulated. In certain embodiments, CD38 is upregulated, CD34 is downregulated, CD11b is upregulated, and CD70 is upregulated. In certain embodiments, CD33 is upregulated, CD34 is downregulated, CD11b is upregulated, and CD70 is upregulated. In certain embodiments, CD38 is upregulated, CD33 is upregulated, CD34 is downregulated, CD11b is upregulated, and CD70 is upregulated.

In further embodiments, step (a) comprises determining an expression level of at least one marker selected from the group consisting of: CD38, CD11b and CD33 of malignant myeloid cells of the human subject. In certain embodiments, CD38 is upregulated, CD33 is upregulated and CD11b is upregulated.

In further embodiments, step (a) comprises determining an expression level of at least one marker selected from the group consisting of: CD45, CD11b and CD117 of malignant myeloid cells of the human subject. In certain embodiments, CD45 is upregulated. In certain embodiments, CD45 is upregulated and CD11b is upregulated. In certain embodiments, CD45 is upregulated and CD117 is downregulated. In certain embodiments, CD45 is upregulated, CD11b is upregulated and CD117 is downregulated.

In further embodiments, step (a) comprises determining an expression level of CD45 and determining the SSC value. In certain embodiments, the cells are characterized as CD45^{bright} and SSC^{high}.

In a particular embodiment, a historical treatment of a BCL-2 inhibitor (e.g., venetoclax) has upregulated CD70 expression on myeloid cells. Myeloid malignancy patients who failed a BCL-2 treatment can then be treated with an antibody or antigen binding fragment thereof that binds to CD70 (e.g., cusatuzumab). Treatment with an antibody or antigen binding fragment thereof that binds to CD70 in turn upregulates BCL-2 expression on myeloid cells. So treatment with a BCL-2 inhibitor (e.g., venetoclax) and an antibody or antigen binding fragment thereof that binds to CD70 (e.g., cusatuzumab) have a reciprocal effect in myeloid malignancy patients and improve treatment responses in these patients. In a particular embodiment, an anti-CD70 antibody or CD70-binding fragment thereof is combined (co-administered) with a BCL-2 inhibitor for use in treating a myeloid malignancy in a patient who is resistant to BCL-2 inhibitor treatment. In certain embodiments, step (a) comprises determining a CD70 expression level of malignant myeloid cells of the human subject. The relevant expression level can be determined using any suitable method, including, without limitation, fluorescence-activated cell sorting (FACS) and fluorescence microscopy using detectable (e.g., fluorescently labeled) antibodies specific for CD70.

In certain embodiments, CD70 is upregulated compared to a CD70 expression level as measured before or during a BCL-2 inhibitor treatment. In certain embodiments, the BCL-2 inhibitor treatment comprises treatment with venetoclax. In certain embodiments, the BCL-2 inhibitor treatment comprises treatment with a BCL-2 inhibitor other than venetoclax.

In certain embodiments, the human subject has a clinical history comprising:
(a) treatment with a BCL-2 inhibitor; and
(b) absence of a remission in response to the treatment with the BCL-2 inhibitor. In certain embodiments, the absence of a remission is an absence of a complete remission. In other embodiments, the absence of a remission is an absence of at least a partial remission. In certain embodiments, the historical treatment with the BCL-2 inhibitor is treatment with venetoclax. In certain other embodiments, the historical treatment with the BCL-2 inhibitor is treatment with a BCL-2 inhibitor other than venetoclax.

In certain embodiments, the historical treatment with the BCL-2 inhibitor further comprises treatment with a hypomethylating agent (HMA). Hypomethylating agents inhibit normal methylation of DNA and/or RNA. Nonlimiting examples of hypomethylating agents are azacitidine, decitabine, and guadecitabine.

Azacitidine is an analogue of cytidine, and decitabine is its deoxy derivative. Guadecitabine is a cytidine deaminase-resistant prodrug of decitabine. Azacitidine and decitabine are inhibitors of DNA methyltransferases (DNMT) known to upregulate gene expression by promoter hypomethylation. Such hypomethylation disrupts cell function, thereby resulting in cytotoxic effects.

In certain embodiments, the human subject has a clinical history comprising:
(a) treatment with a BCL-2 inhibitor;
(b) partial or complete remission; and
(c) partial or complete relapse.

In certain embodiments, human subject has a clinical history comprising treatment with a BCL-2 inhibitor; partial remission; and partial relapse.

In certain embodiments, human subject has a clinical history comprising treatment with a BCL-2 inhibitor; partial remission; and complete relapse.

In certain embodiments, human subject has a clinical history comprising treatment with a BCL-2 inhibitor; complete remission; and partial relapse.

In certain embodiments, human subject has a clinical history comprising treatment with a BCL-2 inhibitor; complete remission; and complete relapse.

Further in accordance with these embodiments, in certain embodiments the historical treatment with the BCL-2 inhibitor further comprises treatment with a hypomethylating agent (HMA). In certain embodiments, the historical treatment with the BCL-2 inhibitor is treatment with venetoclax or a pharmaceutically acceptable salt thereof. In certain other embodiments, the historical treatment with the BCL-2 inhibitor is treatment with a BCL-2 inhibitor other than venetoclax or a pharmaceutically acceptable salt thereof. As mentioned above, nonlimiting examples of hypomethylating agents are azacitidine, decitabine, and guadecitabine.

In accordance with each of the foregoing embodiments, in certain embodiments, a hypomethylating agent (HMA) is co-administered with the antibody or antigen binding fragment thereof that binds to CD70. In certain embodiments, the HMA is selected from the group consisting of azacitidine, decitabine, guadecitabine, and any combination thereof. In certain embodiments, the HMA is azacitidine. In certain embodiments, the HMA is decitabine. In certain embodiments, the HMA is guadecitabine.

The HMA can be administered on the same schedule or substantially the same schedule as that of the antibody or antigen binding fragment thereof that binds to CD70, or it can be on a different schedule from that of the antibody or antigen binding fragment thereof that binds to CD70. Moreover, the route of administration of the HMA can be the same route of administration as that of the antibody or antigen binding fragment thereof that binds to CD70, or it can be different from the route of administration of the antibody or antigen binding fragment thereof that binds to CD70.

In accordance with each of the foregoing embodiments, in certain embodiments, a BCL-2 inhibitor is co-administered with the antibody or antigen binding fragment thereof that binds to CD70. The BCL-2 inhibitor can be administered on the same schedule or substantially the same schedule as that of the antibody or antigen binding fragment thereof that binds to CD70, or it can be on a different schedule from that of the antibody or antigen binding fragment thereof that binds to CD70. Moreover, the route of administration of the BCL-2 inhibitor can be the same route of administration as that of the antibody or antigen binding fragment thereof that binds to CD70, or it can be different from the route of administration of the antibody or antigen binding fragment thereof that binds to CD70.

In certain embodiments, the antibody or antigen binding fragment thereof that binds to CD70 is co-administered with a BCL-2 inhibitor and a hypomethylating agent (HMA). In certain embodiments, the antibody that binds to CD70 is cusatuzumab. In certain embodiments, the BCL-2 inhibitor is venetoclax or a pharmaceutically acceptable salt thereof. In certain embodiments, the HMA is azacitidine. In certain embodiments, cusatuzumab is co-administered with venetoclax and azacitidine.

In accordance with each of the foregoing embodiments, in certain embodiments, venetoclax or a pharmaceutically acceptable salt thereof is co-administered with the antibody or antigen binding fragment thereof that binds to CD70. The venetoclax or a pharmaceutically acceptable salt thereof can be administered on the same schedule or substantially the same schedule as that of the antibody or antigen binding fragment thereof that binds to CD70, or it can be on a different schedule from that of the antibody or antigen binding fragment thereof that binds to CD70. Moreover, the route of administration of the venetoclax or a pharmaceutically acceptable salt thereof can be the same route of administration as that of the antibody or antigen binding fragment thereof that binds to CD70, or it can be different from the route of administration of the antibody or antigen binding fragment thereof that binds to CD70.

In certain embodiments, the antibody or antibody binding fragment that binds to CD70 comprises HCDR1, HCDR2, HCDR3, LCDR1, LCDR2, and LCDR3, wherein
the amino acid sequence of HCDR1 consists of SEQ ID NO: 1;
the amino acid sequence of HCDR2 consists of SEQ ID NO: 2;
the amino acid sequence of HCDR3 consists of SEQ ID NO: 3;
the amino acid sequence of LCDR1 consists of SEQ ID NO: 4;
the amino acid sequence of LCDR2 consists of SEQ ID NO: 5; and
the amino acid sequence of LCDR3 consists of SEQ ID NO: 6.

In certain embodiments, the antibody or antibody binding fragment that binds to CD70 comprises a variable heavy chain domain (VH) comprising an amino acid sequence at least 90% identical to SEQ ID NO: 7.

In certain embodiments, the antibody or antibody binding fragment that binds to CD70 comprises a variable heavy chain domain (VH) comprising an amino acid sequence at least 91% identical to SEQ ID NO: 7.

In certain embodiments, the antibody or antibody binding fragment that binds to CD70 comprises a variable heavy chain domain (VH) comprising an amino acid sequence at least 92% identical to SEQ ID NO: 7.

In certain embodiments, the antibody or antibody binding fragment that binds to CD70 comprises a variable heavy chain domain (VH) comprising an amino acid sequence at least 93% identical to SEQ ID NO: 7.

In certain embodiments, the antibody or antibody binding fragment that binds to CD70 comprises a variable heavy chain domain (VH) comprising an amino acid sequence at least 94% identical to SEQ ID NO: 7.

In certain embodiments, the antibody or antibody binding fragment that binds to CD70 comprises a variable heavy chain domain (VH) comprising an amino acid sequence at least 95% identical to SEQ ID NO: 7.

In certain embodiments, the antibody or antibody binding fragment that binds to CD70 comprises a variable heavy chain domain (VH) comprising an amino acid sequence at least 96% identical to SEQ ID NO: 7.

In certain embodiments, the antibody or antibody binding fragment that binds to CD70 comprises a variable heavy chain domain (VH) comprising an amino acid sequence at least 97% identical to SEQ ID NO: 7.

In certain embodiments, the antibody or antibody binding fragment that binds to CD70 comprises a variable heavy chain domain (VH) comprising an amino acid sequence at least 98% identical to SEQ ID NO: 7.

In certain embodiments, the antibody or antibody binding fragment that binds to CD70 comprises a variable heavy chain domain (VH) comprising an amino acid sequence at least 99% identical to SEQ ID NO: 7.

In certain embodiments, the antibody or antibody binding fragment that binds to CD70 comprises a variable heavy chain domain (VH) comprising an amino acid sequence 100% identical to SEQ ID NO: 7.

In certain embodiments, the antibody or antibody binding fragment that binds to CD70 comprises a variable light chain domain (VL) comprising an amino acid sequence at least 90% identical to SEQ ID NO: 8.

In certain embodiments, the antibody or antibody binding fragment that binds to CD70 comprises a variable light chain domain (VL) comprising an amino acid sequence 91% identical to SEQ ID NO: 8.

In certain embodiments, the antibody or antibody binding fragment that binds to CD70 comprises a variable light chain domain (VL) comprising an amino acid sequence at least 92% identical to SEQ ID NO: 8.

In certain embodiments, the antibody or antibody binding fragment that binds to CD70 comprises a variable light chain domain (VL) comprising an amino acid sequence at least 93% identical to SEQ ID NO: 8.

In certain embodiments, the antibody or antibody binding fragment that binds to CD70 comprises a variable light chain domain (VL) comprising an amino acid sequence at least 94% identical to SEQ ID NO: 8.

In certain embodiments, the antibody or antibody binding fragment that binds to CD70 comprises a variable light chain domain (VL) comprising an amino acid sequence at least 95% identical to SEQ ID NO: 8.

In certain embodiments, the antibody or antibody binding fragment that binds to CD70 comprises a variable light chain domain (VL) comprising an amino acid sequence at least 96% identical to SEQ ID NO: 8.

In certain embodiments, the antibody or antibody binding fragment that binds to CD70 comprises a variable light chain domain (VL) comprising an amino acid sequence at least 97% identical to SEQ ID NO: 8.

In certain embodiments, the antibody or antibody binding fragment that binds to CD70 comprises a variable light chain domain (VL) comprising an amino acid sequence at least 98% identical to SEQ ID NO: 8.

In certain embodiments, the antibody or antibody binding fragment that binds to CD70 comprises a variable light chain domain (VL) comprising an amino acid sequence at least 99% identical to SEQ ID NO: 8.

In certain embodiments, the antibody or antibody binding fragment that binds to CD70 comprises a variable light chain domain (VL) comprising an amino acid sequence 100% identical to SEQ ID NO: 8.

In certain embodiments, the antibody or antibody binding fragment that binds to CD70 comprises a variable heavy chain domain (VH) comprising an amino acid sequence at least 90% identical to SEQ ID NO: 7 and a variable light chain domain (VL) comprising an amino acid sequence at least 90% identical to SEQ ID NO: 8.

In certain embodiments, the antibody or antibody binding fragment that binds to CD70 comprises a variable heavy chain domain (VH) comprising an amino acid sequence at least 91% identical to SEQ ID NO: 7 and a variable light chain domain (VL) comprising an amino acid sequence at least 91% identical to SEQ ID NO: 8.

In certain embodiments, the antibody or antibody binding fragment that binds to CD70 comprises a variable heavy chain domain (VH) comprising an amino acid sequence at least 92% identical to SEQ ID NO: 7 and a variable light chain domain (VL) comprising an amino acid sequence at least 92% identical to SEQ ID NO: 8.

In certain embodiments, the antibody or antibody binding fragment that binds to CD70 comprises a variable heavy chain domain (VH) comprising an amino acid sequence at least 93% identical to SEQ ID NO: 7 and a variable light chain domain (VL) comprising an amino acid sequence at least 93% identical to SEQ ID NO: 8.

In certain embodiments, the antibody or antibody binding fragment that binds to CD70 comprises a variable heavy chain domain (VH) comprising an amino acid sequence at least 94% identical to SEQ ID NO: 7 and a variable light chain domain (VL) comprising an amino acid sequence at least 94% identical to SEQ ID NO: 8.

In certain embodiments, the antibody or antibody binding fragment that binds to CD70 comprises a variable heavy chain domain (VH) comprising an amino acid sequence at least 95% identical to SEQ ID NO: 7 and a variable light chain domain (VL) comprising an amino acid sequence at least 95% identical to SEQ ID NO: 8.

In certain embodiments, the antibody or antibody binding fragment that binds to CD70 comprises a variable heavy chain domain (VH) comprising an amino acid sequence at least 96% identical to SEQ ID NO: 7 and a variable light chain domain (VL) comprising an amino acid sequence at least 96% identical to SEQ ID NO: 8.

In certain embodiments, the antibody or antibody binding fragment that binds to CD70 comprises a variable heavy chain domain (VH) comprising an amino acid sequence at least 97% identical to SEQ ID NO: 7 and a variable light chain domain (VL) comprising an amino acid sequence at least 97% identical to SEQ ID NO: 8.

In certain embodiments, the antibody or antibody binding fragment that binds to CD70 comprises a variable heavy chain domain (VH) comprising an amino acid sequence at least 98% identical to SEQ ID NO: 7 and a variable light chain domain (VL) comprising an amino acid sequence at least 98% identical to SEQ ID NO: 8.

In certain embodiments, the antibody or antibody binding fragment that binds to CD70 comprises a variable heavy chain domain (VH) comprising an amino acid sequence at least 99% identical to SEQ ID NO: 7 and a variable light chain domain (VL) comprising an amino acid sequence at least 99% identical to SEQ ID NO: 8.

In certain embodiments, the antibody or antibody binding fragment that binds to CD70 comprises a variable heavy chain domain (VH) comprising an amino acid sequence 100% identical to SEQ ID NO: 7 and a variable light chain domain (VL) comprising an amino acid sequence 100% identical to SEQ ID NO: 8.

In certain embodiments, the amino acid sequence which is at least 90% identical to the VH consisting of SEQ ID NO: 7 comprises HCDR1, HCDR2, and HCDR3, wherein
the amino acid sequence of HCDR1 consists of SEQ ID NO: 1;
the amino acid sequence of HCDR2 consists of SEQ ID NO: 2; and
the amino acid sequence of HCDR3 consists of SEQ ID NO: 3; and
the amino acid sequence which is at least 90% identical to the VL consisting of SEQ ID NO: 8 comprises LCDR1, LCDR2, and LCDR3, wherein
the amino acid sequence of LCDR1 consists of SEQ ID NO: 4;
the amino acid sequence of LCDR2 consists of SEQ ID NO: 5; and
the amino acid sequence of LCDR3 consists of SEQ ID NO: 6.

In certain embodiments, the method may further comprise administering one or more additional therapeutic agents, for example at least one additional anti-cancer agent, preferably an agent for the treatment of a myeloid malignancy. In certain embodiments, the additional anti-cancer agent is an agent for the treatment of acute myeloid leukemia (AML).

In certain embodiments, the CD70 antibody or antigen binding fragment thereof is administered at a dose in the range of 0.1-25 mg/kg, preferably 10 mg/kg. Alternatively or in addition, the BCL-2 inhibitor, preferably venetoclax or pharmaceutically acceptable salt thereof, may be administered in a dose in the range 100 mg-600 mg. In preferred embodiments, the methods described herein comprise administering a combination additionally comprising azacitidine wherein the azacitidine is administered at a dose of 75 mg/m². In further preferred embodiments, the methods described herein comprise administering a combination additionally comprising decitabine wherein the decitabine is administered at a dose of 20 mg/m².

In certain embodiments, the methods further comprise monitoring of the patient's blast count. The patient's peripheral blood and/or bone marrow count may be reduced, for example reduced to less than 25%, for example reduced to 5%, for example reduced to less than 5%, for example reduced to minimal residual disease levels, for example reduced to undetectable levels. In certain embodiments, the bone marrow blast count is reduced to between 5% and 25% and the bone marrow blast percentage is reduced by more than 50% as compared to pretreatment.

In certain embodiments, the methods induce a partial remission. In certain embodiments, the methods induce a complete remission, optionally with platelet recovery and/or neutrophil recovery. The methods may induce transfusion independence of red blood cells or platelets, or both, for 8 weeks or longer, 10 weeks or longer, 12 weeks or longer. In certain embodiments, the methods reduce the mortality rate after a 30-day period or after a 60-day period.

In certain embodiments, the methods increase survival. For example, the methods may increase survival relative to the standard of care agent or agents used to treat the particular myeloid malignancy being treated with the combination. The methods may induce a minimal residual disease status that is negative.

In certain embodiments, the methods further comprise a step of subjecting the subject to a bone marrow transplantation. Alternatively or in addition, the methods may further comprise a step of administering one or more additional anti-cancer agents. The one or more additional cancer agents may be selected from any agents suitable for the treatment of myeloid malignancies, preferably AML. Preferred agents may be selected from selectin inhibitors (e.g., GMI-1271); FMS-like tyrosine kinase receptor 3 (FLT3) inhibitors (e.g., midostaurin); cyclin-dependent kinase inhibitors; aminopeptidase inhibitors; JAK/STAT inhibitors; cytarabine; anthracycline compounds (e.g., daunorubicin, idarubicin); doxorubicin; hydroxyurea; Vyxeos; IDH1 or IDH2 inhibitors such as Idhifa (or Enasidenib) or Tibsovo (or ivosidenib); Smoothened inhibitors such as Glasdegib, BET bromodomain inhibitors, CD123 or CD33 targeting agents, HDAC inhibitors, LSC targeting agents, AML bone marrow niche targeting agents, and NEDD8-activating enzyme inhibitors such as Pevonedistat.

The CD70 antibodies or antigen binding fragments in accordance with the methods described herein may be formulated using any suitable pharmaceutical carriers, adjuvants and/or excipients. Techniques for formulating antibodies for human therapeutic use are well known in the art and are reviewed, for example, in Wang et al. (2007) Journal of Pharmaceutical Sciences, 96:1-26, the contents of which are incorporated herein in their entirety. Pharmaceutically acceptable excipients that may be used to formulate the antibody compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol, wool fat and hyaluronidases (for example PH20 enzyme).

The BCL-2 inhibitor (preferably venetoclax or a pharmaceutically acceptable salt thereof) may be formulated using any suitable pharmaceutical carriers, adjuvants and/or excipients. Suitable agents include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

It has been found that CD70 antibodies, particularly ARGX-110, are effective for the treatment of myeloid malignancy, particularly AML, at relatively low dose. Therefore, in certain embodiments of all methods of the invention the CD70 antibody or antigen binding fragment thereof is administered at a dose in the range from 0.1 mg/kg to 30 mg/kg per dose. In certain embodiments of all methods of the invention the CD70 antibody or antigen binding fragment thereof is administered at a dose in the range from 0.1 mg/kg to 25 mg/kg per dose, for example in the range of from 0.1 mg/kg to 20 mg/kg. In certain embodiments, the CD70 antibody or antigen binding fragment thereof is administered at a dose in the range from 1 mg/kg to 20 mg/kg per dose. Ranges described herein include the end points of the range unless indicated otherwise—for example, administration at a dose in the range of 0.1-25 mg/kg includes administration at a dose of 0.1 mg/kg and administration at a dose of 25 mg/kg, as well as all doses between the two end points.

In certain embodiments of methods of the invention, the CD70 antibody or antigen binding fragment thereof is administered at a dose in the range from 0.1-15 mg/kg. In certain embodiments the CD70 antibody or antigen binding fragment thereof is administered at a dose in the range from 0.5-2 mg/kg. In certain embodiments the CD70 antibody or antigen binding fragment thereof is administered at a dose of 1 mg/kg, 3 mg/kg, 10 mg/kg, or 20 mg/kg. In certain preferred embodiments the CD70 antibody or antigen binding fragment thereof is administered at a dose of 1mg/kg. In certain preferred embodiments the CD70 antibody or antigen binding fragment thereof is administered at a dose of 10 mg/kg.

In certain embodiments, multiple doses of the CD70 antibody or antigen binding fragment are administered. In certain such embodiments, each dose of the CD70 antibody or antigen-binding fragment thereof is separated by 10-20 days, optionally 12-18 days. In certain embodiments each dose of anti-CD70 antibody is separated by 14-17 days.

The BCL-2 inhibitor, preferably venetoclax or pharmaceutically acceptable salt thereof, may be dosed according to any regimen determined to be effective for the compound. The FDA prescribing information for use of VENCLEXTA® in treating AML proposes a dosing schedule having a ramp-up phase followed by a maintenance phase. In situations where VENCLEXTA® is prescribed in combination with azacitidine or decitabine, a dosing schedule is recommended consisting of: 100 mg VENCLEXTA® on day 1; 200 mg VENCLEXTA® on day 2; 400 mg VENCLEXTA® on day 3; and 400 mg VENCLEXTA® in combination with 75 mg/m² azacitidine or 20 mg/m² decitabine daily thereafter until disease progression or unacceptable toxicity is observed. In situations where VENCLEXTA® is prescribed in combination with low-dose cytarabine, a dosing schedule is recommended consisting of: 100 mg VENCLEXTA® on day 1; 200 mg VENCLEXTA® on day 2; 400 mg VENCLEXTA® on day 3; and 600 mg VENCLEXTA® in combination with 20 mg/m² daily thereafter until disease progression or unacceptable toxicity is observed.

In certain embodiments, each dose, for example oral dose, of the venetoclax or pharmaceutically acceptable salt thereof is in the range from 100 mg-600 mg. In certain embodiments, the venetoclax or pharmaceutically acceptable salt thereof is dosed daily at 400 mg. In certain embodiments, the venetoclax or pharmaceutically acceptable salt thereof is dosed daily at 600 mg. As described above, the daily fixed-dosing of venetoclax may be preceded by a ramp-up period, for example 3 days, wherein increasing doses of venetoclax are administered to the patient until the maintenance daily dose is reached.

In certain embodiments, the methods described herein involve monitoring the patient's blast count i.e. the number of blast cells. As used herein, “blast cells” or “blasts” refer to myeloblasts or myeloid blasts which are the myeloid progenitor cells within the bone marrow. In healthy individuals, blasts are not found in the peripheral blood circulation and there should be less than 5% blast cells in the bone marrow. In subjects with myeloid malignancies, particularly AML and MDS, there is increased production of abnormal blasts with disrupted differentiation potential, and the overproduction of these abnormal blasts can be detected by monitoring the patient's blast count in the peripheral blood circulation or the bone marrow or both.

The proportion of blast cells in the bone marrow or peripheral blood can be assessed by methods known in the art, for example flow cytometric or cell morphologic assessment of cells obtained from a bone marrow biopsy of the subject, or a peripheral blood smear. The proportion of blasts is determined versus total cells in the sample. For example, flow cytometry can be used to determine the proportion of blast cells using the number of CD45^{dim}, SSC^{low} cells relative to total cell number. By way of further example, cell morphological assessment can be used to determine the number of morphologically identified blasts relative to the total number of cells in the field of view being examined.

In certain embodiments are provided methods for reducing the proportion of blasts cells in the bone marrow to less than 25%, less than 20%, for example less than 10%. In certain embodiments are provided methods for reducing the proportion of blasts cells in the bone marrow to less than 5%. In certain embodiments are provided methods for reducing the proportion of blast cells in the bone marrow to between about 5% and about 25%, wherein the bone marrow blast cell percentage is also reduced by more than 50% as compared with the bone marrow blast cell percentage prior to performing the method (or pretreatment).

In certain embodiments are provided methods for reducing the proportion of blasts cells in the peripheral blood to less than 25%, less than 20%, for example less than 10%. In certain embodiments are provided methods for reducing the proportion of blasts cells in the peripheral blood to less than 5%. In certain embodiments are provided methods for reducing the proportion of blast cells in the peripheral blood to between about 5% and about 25%, wherein the peripheral blood blast cell percentage is also reduced by more than 50% as compared with the peripheral blast cell percentage prior to performing the method (or pretreatment).

For clinical determination of blast cell percentage, typically cell morphological (also known as cytomorphology) assessment is preferred.

In particular embodiments, the methods described herein induce a complete response. In the context of AML treatment, a complete response or “complete remission” is defined as: bone marrow blasts <5%; absence of circulating blasts and blasts with Auer rods; absence of extramedullary disease; ANC ≥1.0×10⁹/L (1000 μL); platelet count ≥100×10⁹/L (100,000 μL), see Döhner et al. (2017) Blood 129(4): 424-447.

The methods may achieve a complete response with platelet recovery i.e. a response wherein the platelet count is ≥100×10⁹/L (100,000/μL). The methods may achieve a complete response with neutrophil recovery i.e. a response wherein the neutrophil count is ≥1.0×10⁹/L (1000/μL). Alternatively or in addition, the methods may induce a transfusion independence of red blood cells or platelets, or both, for 8 weeks or longer, 10 weeks or longer, 12 weeks or longer.

In particular embodiments, the methods described herein induce a minimal or measurable residual disease (or MRD) status that is negative, see Schuurhuis et al. (2018) Blood. 131(12): 1275-1291.

In certain embodiments, the methods described herein induce a complete response without minimal residual disease (CRMRD-), see Döhner et al. (2017) Blood 129(4): 424-447.

The method may achieve a partial response or induce partial remission. In the context of AML treatment, a partial response or partial remission includes a decrease of the bone marrow blast percentage of 5% to 25% and a decrease of pretreatment bone marrow blast percentage by at least 50%, see Döhner et al. Ibid.

The methods described herein may increase survival. The term “survival” as used herein may refer to overall survival, 1-year survival, 2-year survival, 5-year survival, event-free survival, progression-free survival. The methods described herein may increase survival as compared with the gold-standard treatment for the particular disease or condition to be treated. The gold-standard treatment may also be identified as the best practice, the standard of care, the standard medical care or standard therapy. For any given disease, there may be one or more gold-standard treatments depending on differing clinical practice, for example in different countries. The treatments already available for myeloid malignancies are varied and include chemotherapy, radiation therapy, stem cell transplant and certain targeted therapies. Furthermore, clinical guidelines in both the US and Europe govern the standard treatment of myeloid malignancies, for example AML, see O'Donnell et al. (2017) Journal of the National Comprehensive Cancer Network 15(7): 926-957 and Döhner et al. (2017) Blood 129(4): 424-447, both incorporated by reference.

The methods of the present invention may increase or improve survival relative to patients undergoing any of the standard treatments for myeloid malignancy.

The methods described herein may include a further step of subjecting the patient or subject to a bone marrow transplant. The methods described herein may also be used to prepare a patient or subject having a myeloid malignancy for a bone marrow transplantation. As described above, the methods of the present invention may be carried out so as to reduce the absolute or relative numbers of blast cells in the bone marrow or peripheral blood. In certain embodiments, the methods are carried out so as to reduce the blast cell count in the bone marrow and/or peripheral blood prior to transplant. The methods may be used to reduce the blast cell count to less than 5% to prepare the patient or subject for a bone marrow transplant.

An aspect of the invention is a method of identifying and treating a patient to be treated with an anti-CD70 antibody or antigen-binding fragment thereof, wherein the patient has a myeloid malignancy, the method comprising the steps of:
(i) measuring the myeloid differentiation status of the patient;
(ii) determining whether the patient has differentiated monocytic AML, wherein a patient having differentiated monocytic AML is identified as a patient to be treated with the anti-CD70 antibody or CD70-binding fragment thereof; and
(iii) administering the anti-CD70 antibody or CD70-binding fragment thereof to the patient identified as a patient to be treated with the anti-CD70 antibody or CD70-binding fragment thereof.

In certain embodiments, step (i) and (ii) are performed on a sample obtained from the patient with a myeloid malignancy.

In certain embodiments, a bone marrow sample of the patient comprises CD45^{bright}/SSC^{high}/CD38⁺/CD34⁻/CD33⁺/CD11b⁺/CD70⁺ phenotype cells or CD45^{bright}/SSC^{high}/CD34⁻/CD117⁻/CD11b⁺/CD68⁺/CD14⁺/CD64⁺ phenotype cells.

### C. Medical Uses

In a further aspect, the invention provides an antibody or antigen binding fragment thereof that binds to CD70 for use in therapy. In particular, the antibody or antigen binding fragment thereof that binds to CD70 is for use in treating a myeloid malignancy in a human subject. In particular, the antibody or antigen binding fragment thereof that binds to CD70 is for use in treating a myeloid malignancy in a human subject who is resistant to BCL-2 inhibitor treatment.

In particular embodiments, the human subject is identified as having differentiated monocytic AML on the basis of differential expression levels of one or more markers.

In particular embodiments, the treatment is preceded by a selection comprising the steps of:
(i) measuring the myeloid differentiation status of the human subject, and
(ii) determining whether the human subject has differentiated monocytic AML, and
wherein a therapeutically effective dose of the anti-CD70 antibody or anti-CD70-binding fragment thereof is administered to said human subject having differentiated monocytic AML.

In a further aspect, the present invention provides an antibody or antigen binding fragment thereof that binds to CD70 for use in a method of treating a myeloid malignancy in a human subject, said method comprising the steps of:
(a) selecting a human subject having a myeloid malignancy that has a reduced sensitivity or is refractory to a BCL-2 inhibitor; and
(b) administering to the human subject an antibody or antigen binding fragment thereof that binds to CD70.

In certain embodiments, there is provided an antibody or antigen binding fragment thereof that binds to CD70 for use in a method of treating a myeloid malignancy in a human subject as described herein, wherein the antibody or antigen binding fragment thereof is administered in combination with a BCL-2 inhibitor.

In certain embodiments, there is provided an antibody or antigen binding fragment thereof that binds to CD70 for use in a method of treating a myeloid malignancy in a human subject as described herein, wherein the antibody or antigen binding fragment thereof is administered in combination with a hypomethylating agent (HMA).

In certain embodiments, there is provided an antibody or antigen binding fragment thereof that binds to CD70 for use in a method of treating a myeloid malignancy in a human subject as described herein, wherein the antibody or antigen binding fragment thereof is administered in combination with a BCL-2 inhibitor and a hypomethylating agent (HMA).

In certain embodiments, there is provided a combination of an antibody or antigen binding fragment thereof that binds to CD70 and a BCL-2 inhibitor for use in a method of treating a myeloid malignancy in a human subject as described herein.

In certain embodiments, there is provided a combination of an antibody or antigen binding fragment thereof that binds to CD70 and a hypomethylating agent (HMA) for use in a method of treating a myeloid malignancy in a human subject as described herein.

In certain embodiments, there is provided a combination of an antibody or antigen binding fragment thereof that binds to CD70, a BCL-2 inhibitor and a hypomethylating agent (HMA) for use in a method of treating a myeloid malignancy in a human subject as described herein.

In certain embodiments, the antibody that binds to CD70 is cusatuzumab. In certain embodiments, the BCL-2 inhibitor is venetoclax or a pharmaceutically acceptable salt thereof. In certain embodiments, the HMA is azacitidine. In certain embodiments, the combination is cusatuzumab, venetoclax and azacitidine.

In certain embodiments, the myeloid malignancy is AML. In certain embodiments, the myeloid malignancy is monocytic AML. In certain embodiments, the human subject is identified as having differentiated monocytic AML on the basis of differential expression levels of one or more markers.

In certain embodiments, the human subject is identified as having differentiated monocytic AML on the basis of differential expression level(s) of at least one marker selected from the group consisting of: BCL-2, CD117, CD11b, CD68, CD64, BCL2A1, and MCL1, of malignant myeloid cells of the human subject. Relevant expression levels can be determined using any suitable method, including, without limitation, fluorescence-activated cell sorting (FACS), fluorescence microscopy using detectable (e.g., fluorescently labeled) antibodies specific for the relevant cell surface molecule(s) and mRNA expression analysis. In certain embodiments, the human subject is identified as having differentiated monocytic AML on the basis of differential expression level(s) of at least one marker selected from the group consisting of: BCL-2, CD117, CD11b, CD68, CD64, BCL2A1, and MCL1, of malignant myeloid cells of the human subject. In certain embodiments, the human subject is identified as having differentiated monocytic AML on the basis of differential expression level(s) of at least one marker selected from the group consisting of: BCL-2, CD117, CD11b, CD68, CD64, BCL2A1, and MCL1, of malignant myeloid cells of the human subject. In certain embodiments, the human subject is identified as having differentiated monocytic AML on the basis of an expression level of BCL-2 of malignant myeloid cells of the human subject. In certain embodiments, the human subject is identified as having differentiated monocytic AML on the basis of an expression level of CD117 of malignant myeloid cells of the human subject. In certain embodiments, the human subject is identified as having differentiated monocytic AML on the basis of an expression level of CD11b of malignant myeloid cells of the human subject. In certain embodiments, the human subject is identified as having differentiated monocytic AML on the basis of an expression level of CD68 of malignant myeloid cells of the human subject. In certain embodiments, the human subject is identified as having differentiated monocytic AML on the basis of an expression level of CD64 of malignant myeloid cells of the human subject. In certain embodiments, the human subject is identified as having differentiated monocytic AML on the basis of an expression level of BCL2A1 of malignant myeloid cells of the human subject.

In certain embodiments, the human subject is identified as having differentiated monocytic AML on the basis of an expression level of MCLI of malignant myeloid cells of the human subject.

In certain embodiments, the expression level(s) of at least one of BCL-2 and CD117 is downregulated, and at least one of CD11b, CD68, CD64, CD70, BCL2A1, and MCL1 is upregulated. In certain embodiments, the expression level of BCL-2 is downregulated and the expression level of CD11b is upregulated. In certain embodiments, the expression level of BCL-2 is downregulated and the expression level of CD68 is upregulated. In certain embodiments, the expression level of BCL-2 is downregulated and the expression level of CD64 is upregulated. In certain embodiments, the expression level of BCL-2 is downregulated and the expression level of CD70 is upregulated. In certain embodiments, the expression level of BCL-2 is downregulated and the expression level of BCL2A1 is upregulated. In certain embodiments, the expression level of BCL-2 is downregulated and the expression level of MCLI is upregulated. In certain embodiments, the expression level of CD117 is downregulated and the expression level of CD11b is upregulated. In certain embodiments, the expression level of CD117 is downregulated and the expression level of CD68 is upregulated. In certain embodiments, the expression level of CD117 is downregulated and the expression level of CD64 is upregulated. In certain embodiments, the expression level of CD117 is downregulated and the expression level of CD70 is upregulated. In certain embodiments, the expression level of CD117 is downregulated and the expression level of BCL2A1 is upregulated. In certain embodiments, the expression level of CD117 is downregulated and the expression level of MCL 1 is upregulated.

In further embodiments, the human subject is identified as having differentiated monocytic AML on the basis of an expression level of at least one marker selected from the group consisting of: CD117, CD11b and CD68. In certain embodiments, the human subject is identified as having differentiated monocytic AML on the basis of an expression level of CD117 of malignant myeloid cells of the human subject. In certain embodiments, the human subject is identified as having differentiated monocytic AML on the basis of an expression level of CD11b of malignant myeloid cells of the human subject. In certain embodiments, the human subject is identified as having differentiated monocytic AML on the basis of an expression level of CD68 of malignant myeloid cells of the human subject. In certain embodiments, the expression level of CD117 is downregulated. In certain embodiments, the expression level of CD11b is upregulated. In certain embodiments, the expression level of CD68 is upregulated. In certain embodiments, the expression level of CD11b is upregulated and the expression level of CD68 is upregulated. In certain embodiments, the expression level of CD117 is downregulated, the expression level of CD11b is upregulated and the expression level of CD68 is upregulated.

In further embodiments, the human subject is identified as having differentiated monocytic AML on the basis of an expression level of at least one marker selected from the group consisting of: CD64, CD34, CD117, CD11b, CD68 and CD14 of malignant myeloid cells of the human subject. In certain embodiments, the human subject is identified as having differentiated monocytic AML on the basis of an expression level of CD64 of malignant myeloid cells of the human subject. In certain embodiments, the human subject is identified as having differentiated monocytic AML on the basis of an expression level of CD34 of malignant myeloid cells of the human subject. In certain embodiments, the human subject is identified as having differentiated monocytic AML on the basis of an expression level of CD117 of malignant myeloid cells of the human subject. In certain embodiments, the human subject is identified as having differentiated monocytic AML on the basis of an expression level of CD11b of malignant myeloid cells of the human subject. In certain embodiments, the human subject is identified as having differentiated monocytic AML on the basis of an expression level of CD68 of malignant myeloid cells of the human subject. In certain embodiments, the human subject is identified as having differentiated monocytic AML on the basis of an expression level of CD14 of malignant myeloid cells of the human subject. In certain embodiments, the expression level of CD64 is upregulated. In certain embodiments, the expression level of CD34 is downregulated. In certain embodiments, the expression level of CD117 is downregulated. In certain embodiments, the expression level of CD11b is upregulated. In certain embodiments, the expression level of CD68 is upregulated. In certain embodiments, the expression level of CD14 is upregulated. In certain embodiments, the expression level of CD64 is upregulated and the expression level of CD34 is downregulated. In certain embodiments, the expression level of CD64 is upregulated and the expression level of CD117 is downregulated. In certain embodiments, the expression level of CD64 is upregulated and the expression level of CD11b is upregulated. In certain embodiments, the expression level of CD64 is upregulated and the expression level of CD68 is upregulated. In certain embodiments, the expression level of CD64 is upregulated and the expression level of CD14 is upregulated. In certain embodiments, the expression level of CD34 is downregulated and the expression level of CD117 is downregulated. In certain embodiments, the expression level of CD34 is downregulated and the expression level of CD11b is upregulated. In certain embodiments, the expression level of CD34 is downregulated and the expression level of CD68 is upregulated. In certain embodiments, the expression level of CD34 is downregulated and the expression level of CD14 is upregulated. In certain embodiments, the expression level of CD117 is downregulated and the expression level of CD14 is upregulated. In certain embodiments, the expression level of CD68 is upregulated and the expression level of CD14 is upregulated. In certain embodiments, the expression level of CD64 is upregulated, the expression level of CD34 is downregulated and the expression level of CD117 is downregulated. In certain embodiments, the expression level of CD64 is upregulated, the expression level of CD34 is downregulated and the expression level of CD11b is upregulated. In certain embodiments, the expression level of CD64 is upregulated, the expression level of CD34 is downregulated and the expression level of CD68 is upregulated. In certain embodiments, the expression level of CD64 is upregulated, the expression level of CD34 is downregulated and the expression level of CD14 is upregulated. In certain embodiments, the expression level of CD64 is upregulated, the expression level of CD117 is downregulated and the expression level of CD14 is upregulated. In certain embodiments, the expression level of CD64 is upregulated, the expression level of CD68 is upregulated and the expression level of CD14 is upregulated. In certain embodiments, the expression level of CD34 is downregulated, the expression level of CD117 is downregulated and the expression level of CD11b is upregulated. In certain embodiments, the expression level of CD34 is downregulated, the expression level of CD117 is downregulated and the expression level of CD68 is upregulated. In certain embodiments, the expression level of CD34 is downregulated, the expression level of CD11b is upregulated and the expression level of CD68 is upregulated. In certain embodiments, the expression level of CD34 is downregulated, the expression level of CD117 is downregulated and the expression level of CD14 is upregulated. In certain embodiments, the expression level of CD34 is downregulated, the expression level of CD11b is upregulated and the expression level of CD14 is upregulated. In certain embodiments, the expression level of CD117 is downregulated, the expression level of CD11b is upregulated and the expression level of CD14 is upregulated. In certain embodiments, the expression level of CD34 is downregulated, the expression level of CD68 is upregulated and the expression level of CD14 is upregulated. In certain embodiments, the expression level of CD117 is downregulated, the expression level of CD68 is upregulated and the expression level of CD14 is upregulated. In certain embodiments, the expression level of CD11b is upregulated, the expression level of CD68 is upregulated and the expression level of CD14 is upregulated. In certain embodiments, the expression level of CD64 is upregulated, the expression level of CD34 is downregulated, the expression level of CD117 is downregulated and the expression level of CD11b is upregulated. In certain embodiments, the expression level of CD64 is upregulated, the expression level of CD34 is downregulated, the expression level of CD117 is downregulated and the expression level of CD68 is upregulated. In certain embodiments, the expression level of CD64 is upregulated, the expression level of CD34 is downregulated, the expression level of CD11b is upregulated and the expression level of CD68 is upregulated. In certain embodiments, the expression level of CD64 is upregulated, the expression level of CD34 is downregulated, the expression level of CD117 is downregulated and the expression level of CD14 is upregulated. In certain embodiments, the expression level of CD64 is upregulated, the expression level of CD34 is downregulated, the expression level of CD11b is upregulated and the expression level of

CD14 is upregulated. In certain embodiments, the expression level of CD64 is upregulated, the expression level of CD117 is downregulated, the expression level of CD11b is upregulated and the expression level of CD14 is upregulated. In certain embodiments, the expression level of CD64 is upregulated, the expression level of CD34 is downregulated, the expression level of CD68 is upregulated and the expression level of CD14 is upregulated. In certain embodiments, the expression level of CD64 is upregulated, the expression level of CD117 is downregulated, the expression level of CD68 is upregulated and the expression level of CD14 is upregulated. In certain embodiments, the expression level of CD64 is upregulated, the expression level of CD11b is upregulated, the expression level of CD68 is upregulated and the expression level of CD14 is upregulated. In certain embodiments, the expression level of CD34 is downregulated, the expression level of CD117 is downregulated, the expression level of CD11b is upregulated and the expression level of CD68 is upregulated. In certain embodiments, the expression level of CD34 is downregulated, the expression level of CD117 is downregulated, the expression level of CD11b is upregulated and the expression level of CD14 is upregulated. In certain embodiments, the expression level of CD34 is downregulated, the expression level of CD117 is downregulated, the expression level of CD68 is upregulated and the expression level of CD14 is upregulated. In certain embodiments, the expression level of CD34 is downregulated, the expression level of CD11b is upregulated, the expression level of CD68 is upregulated and the expression level of CD14 is upregulated. In certain embodiments, the expression level of CD117 is downregulated, the expression level of CD11b is upregulated, the expression level of CD68 is upregulated and the expression level of CD14 is upregulated. In certain embodiments, the expression level of CD64 is upregulated, the expression level of CD34 is downregulated, the expression level of CD117 is downregulated, the expression level of CD11b is upregulated and the expression level of CD68 is upregulated. In certain embodiments, the expression level of CD64 is upregulated, the expression level of CD34 is downregulated, the expression level of CD117 is downregulated, the expression level of CD11b is upregulated and the expression level of CD14 is upregulated. In certain embodiments, the expression level of CD64 is upregulated, the expression level of CD34 is downregulated, the expression level of CD117 is downregulated, the expression level of CD68 is upregulated and the expression level of CD14 is upregulated. In certain embodiments, the expression level of CD64 is upregulated, the expression level of CD34 is downregulated, the expression level of CD11b is upregulated, the expression level of CD68 is upregulated and the expression level of CD14 is upregulated. In certain embodiments, the expression level of CD64 is upregulated, the expression level of CD117 is downregulated, the expression level of CD11b is upregulated, the expression level of CD68 is upregulated and the expression level of CD14 is upregulated. In certain embodiments, the expression level of CD34 is downregulated, the expression level of CD117 is downregulated, the expression level of CD11b is upregulated, the expression level of CD68 is upregulated and the expression level of CD14 is upregulated. In certain embodiments, the expression level of CD64 is upregulated, the expression level of CD34 is downregulated, the expression level of CD117 is downregulated, the expression level of CD11b is upregulated, the expression level of CD68 is upregulated and the expression level of CD14 is upregulated.

In further embodiments, the human subject is identified as having differentiated monocytic AML on the basis of an expression level of at least one marker selected from the group consisting of: CD34, CD38, CD11b, CD33 and CD70 of malignant myeloid cells of the human subject. In certain embodiments, the human subject is identified as having differentiated monocytic AML on the basis of an expression level of CD34 of malignant myeloid cells of the human subject. In certain embodiment, the human subject is identified as having differentiated monocytic AML on the basis of an expression level of CD38 of malignant myeloid cells of the human subject. In certain embodiments, the human subject is identified as having differentiated monocytic AML on the basis of an expression level of CD11b of malignant myeloid cells of the human subject. In certain embodiments, the human subject is identified as having differentiated monocytic AML on the basis of an expression level of CD33 of malignant myeloid cells of the human subject. In certain embodiments, the human subject is identified as having differentiated monocytic AML on the basis of an expression level of CD70 of malignant myeloid cells of the human subject. In certain embodiments, the expression level of CD34 is downregulated. In certain embodiments, the expression level of CD38 is upregulated. In certain embodiments, the expression level of CD11b is upregulated. In certain embodiments, the expression level of CD33 is upregulated. In certain embodiments, the expression level of CD70 is upregulated. In certain embodiments, the expression level of CD34 is downregulated and the expression level of CD38 is upregulated. In certain embodiments, the expression level of CD34 is downregulated and the expression level of CD33 is upregulated. In certain embodiments, the expression level of CD34 is downregulated and the expression level of CD70 is upregulated. In certain embodiments, the expression level of CD38 is upregulated and the expression level of CD33 is upregulated. In certain embodiments, the expression level of CD38 is upregulated and the expression level of CD11b is upregulated. In certain embodiments, the expression level of CD38 is upregulated and the expression level of CD70 is upregulated. In certain embodiments, the expression level of CD33 is upregulated and the expression level of CD11b is upregulated. In certain embodiments, the expression level of CD33 is upregulated and the expression level of CD70 is upregulated. In certain embodiments, the expression level of CD38 is upregulated, the expression level of CD33 is upregulated and the expression level of CD34 is downregulated. In certain embodiments, the expression level of CD38 is upregulated, the expression level of CD33 is upregulated and the expression level of CD11b is upregulated. In certain embodiments, the expression level of CD38 is upregulated, the expression level of CD34 is downregulated and the expression level of CD11b is upregulated. In certain embodiments, the expression level of CD33 is upregulated, the expression level of CD34 is downregulated and the expression level of CD11b is upregulated. In certain embodiments, the expression level of CD38 is upregulated, the expression level of CD33 is upregulated and the expression level of CD70 is upregulated. In certain embodiments, the expression level of CD38 is upregulated, the expression level of CD34 is downregulated and the expression level of CD70 is upregulated. In certain embodiments, the expression level of CD33 is upregulated, the expression level of CD34 is downregulated and the expression level of CD70 is upregulated. In certain embodiments, the expression level of CD38 is upregulated, the expression level of CD11b is upregulated and the expression level of CD70 is upregulated. In certain embodiments, the expression level of CD33 is upregulated, the expression level of CD11b is upregulated and the expression level of CD70 is upregulated. In certain embodiments, the expression level of CD34 is downregulated, the expression level of CD11b is upregulated and the expression level of CD70 is upregulated. In certain embodiments, the expression level of CD38 is upregulated, the expression level of CD33 is upregulated, the expression level of CD34 is downregulated and the expression level of CD11b is upregulated. In certain embodiments, the expression level of CD38 is upregulated, the expression level of CD33 is upregulated, the expression level of CD34 is downregulated and the expression level of CD70 is upregulated. In certain embodiments, the expression level of CD38 is upregulated, the expression level of CD33 is upregulated, the expression level of CD11b is upregulated and the expression level of CD70 is upregulated. In certain embodiments, the expression level of CD38 is upregulated, the expression level of CD34 is downregulated, the expression level of CD11b is upregulated, and the expression level of CD70 is upregulated. In certain embodiments, the expression level of CD33 is upregulated, the expression level of CD34 is downregulated, the expression level of CD11b is upregulated, and the expression level of CD70 is upregulated. In certain embodiments, the expression level of CD38 is upregulated, the expression level of CD33 is upregulated, the expression level of CD34 is downregulated, the expression level of CD11b is upregulated, and the expression level of CD70 is upregulated.

In further embodiments, the human subject is identified as having differentiated monocytic AML on the basis of an expression level of at least one marker selected from the group consisting of: CD38, CD11b and CD33 of malignant myeloid cells of the human subject. In certain embodiments, the expression level of CD38 is upregulated, the expression level of CD33 is upregulated and the expression level of CD11b is upregulated.

In further embodiments, the human subject is identified as having differentiated monocytic AML on the basis of an expression level of at least one marker selected from the group consisting of: CD45, CD11b and CD117 of malignant myeloid cells of the human subject. In certain embodiments, the expression level of CD45 is upregulated. In certain embodiments, the expression level of CD45 is upregulated and the expression level of CD11b is upregulated. In certain embodiments, the expression level of CD45 is upregulated and the expression level of CD117 is downregulated. In certain embodiments, the expression level of CD45 is upregulated, the expression level of CD11b is upregulated and the expression level of CD117 is downregulated.

In further embodiments, the human subject is identified as having differentiated monocytic AML on the basis of an expression level of CD45 and determining the SSC value. In certain embodiments, the cells are characterized as CD45^{bright} and SSC^{high}.

In a particular embodiment, a historical treatment of a BCL-2 inhibitor (e.g., venetoclax) has upregulated CD70 expression on myeloid cells. Myeloid malignancy patients who failed a BCL-2 treatment can then be treated with an antibody or antigen binding fragment thereof that binds to CD70 (e.g., cusatuzumab). Treatment with an antibody or antigen binding fragment thereof that binds to CD70 in turn upregulates BCL-2 expression on myeloid cells. So treatment with a BCL-2 inhibitor (e.g., venetoclax) and an antibody or antigen binding fragment thereof that binds to CD70 (e.g., cusatuzumab) have a reciprocal effect in myeloid malignancy patients and improve treatment responses in these patients. In a particular embodiment, an anti-CD70 antibody or CD70-binding fragment thereof is combined (co-administered) with a BCL-2 inhibitor for use in treating a myeloid malignancy in a patient who is resistant to BCL-2 inhibitor treatment. In certain embodiments, the CD70 expression level of malignant myeloid cells of the human subject is measured. The relevant expression level can be determined using any suitable method, including, without limitation, fluorescence-activated cell sorting (FACS) and fluorescence microscopy using detectable (e.g., fluorescently labeled) antibodies specific for CD70.

In certain embodiments, a bone marrow sample of the patient comprises CD45^{bright}/SSC^{high}/CD38⁺/CD34⁻/CD33⁺/CD11b⁺/CD70⁺ phenotype cells or CD45^{bright}/SSC^{high}/CD34⁻/CD117⁻/CD11b⁺/CD68⁺/CD14⁺/CD64⁺ phenotype cells.

All embodiments described herein relating to the methods of treatment according to the preceding aspects of the invention (see in particular, Section B) are equally applicable to these further aspects and embodiments of the invention.

### D. Use for Manufacture of a Medicament

In a further aspect, the present invention provides a use of an antibody or antigen binding fragment thereof that binds to CD70 for the manufacture of a medicament. In particular, the medicament is of particular use for the treatment of a myeloid malignancy in a human subject, wherein said subject is identified according to the methods described herein.

In certain embodiments, there is provided a use of a combination of an antibody or antigen binding fragment thereof that binds to CD70 and a BCL-2 inhibitor for the manufacture of a medicament for the treatment of a myeloid malignancy in a human subject as described herein.

In certain embodiments, there is provided a use of a combination of an antibody or antigen binding fragment thereof that binds to CD70 and a hypomethylating agent (HMA) for the manufacture of a medicament for the treatment of a myeloid malignancy in a human subject as described herein.

In certain embodiments, there is provided a combination of an antibody or antigen binding fragment thereof that binds to CD70, a BCL-2 inhibitor and a hypomethylating agent (HMA) for the manufacture of a medicament for the treatment of a myeloid malignancy in a human subject as described herein.

All embodiments described herein relating to the methods of treatment according to the preceding aspects of the invention (see in particular, Section B and Section C) are equally applicable to these further aspects and embodiments of the invention.

### E. Diagnostic Methods

A further aspect of the invention is directed to diagnostic methods. Accordingly, in one aspect, the invention provides a method of identifying a patient to be treated with an anti-CD70 antibody or antigen-binding fragment thereof, wherein the patient has a myeloid malignancy and is selected according to a method comprising the steps of:
(i) measuring the myeloid differentiation status of the patient, and
(ii) determining whether the patient has differentiated monocytic AML,
   wherein a patient having differentiated monocytic AML is identified as a patient to be treated with the anti-CD70 antibody or CD70-binding fragment thereof.

In certain embodiments, the steps (i) and (ii) of the method are measured and determined in a sample obtained from the patient.

As used herein, sample includes any tissue or fluid sample obtainable from a patient with a myeloid malignancy. The sample may be used to determine the myeloid differentiation status of a patient. The sample may contain detectable quantities of a marker, preferably a monocytic cell marker. The term sample includes tissues, cells and biological fluids isolated from a subject, as well as tissues, cells and fluids present within a subject. As such in some embodiments, the methods are for determining the myeloid differentiation status of a patient or detecting markers in vitro. “Fluid” as used herein includes for example saliva, mucus, urine, blood, lymphatic fluid and the like. In some embodiments, the sample comprises blood or a fraction or component of blood such as blood serum, blood plasma, or lymph obtained from the patient with a myeloid malignancy. In other embodiments, the sample comprises bone marrow obtained from the patient with a myeloid malignancy.

Therefore, in a further embodiment, there is provided a method of identifying a patient to be treated with an anti-CD70 antibody or antigen-binding fragment thereof, wherein the patient has a myeloid malignancy and is selected according to a method which comprises the steps of:
(i) measuring the myeloid differentiation status of a sample obtained from the patient with a myeloid malignancy;
(ii) determining whether the sample has differentiated monocytic AML; and
   wherein the presence of differentiated monocytic AML in the sample, identifies the patient from which the sample was obtained, as a patient to be treated with the anti-CD70 antibody or CD70-binding fragment thereof.

In certain embodiments, the myeloid differentiation status is determined according the FAB classification system. The FAB system is a well described and a clinically associated means to segregate patients with AML according to their differentiation status. This system classifies AML according to the type of cell that the leukemia develops from and the how mature the cells are. In certain embodiments, the myeloid differentiation status is AML-M5. In other embodiments, the myeloid differentiation status AML-M4. In other embodiments, the myeloid differentiation status is determined according to the WHO classification system.

In further aspects, the level of differentiated monocytic AML in a sample obtained from a patient with a myeloid malignancy can be compared with the a pre-determined cut-off value for the level of differentiated monocytic AML. This allows an assessment to be made as to whether the level of differentiated monocytic AML in the patient is higher, lower, not higher or not lower than the predetermined cut-off value. Such a comparison allows a decision to be made as to whether or not the patient is selected for treatment with an anti-CD70 antibody or antigen-binding fragment thereof.

In a further aspect, there is provided a method of identifying a patient to be treated with an anti-CD70 antibody or antigen-binding fragment thereof, wherein the patient has a myeloid malignancy and is selected according to a method which comprises the step of:
(i) determining the level of differentiated monocytic AML in a sample obtained from the patient with a myeloid malignancy;
(ii) comparing the level of differentiated monocytic AML in (i) with a predetermined cut-off value for differentiated monocytic AML,
   wherein if the level of differentiated monocytic AML determined in the patient sample is higher than the predetermined cut-off value, the patient is selected for treatment with the anti-CD70 antibody or CD70-binding fragment thereof.

In another aspect, there is provided a method of identifying a patient to be treated with an anti-CD70 antibody or antigen-binding fragment thereof, wherein the patient has a myeloid malignancy and is selected according to a method which comprises the step of:
(i) determining the level of differentiated monocytic AML in a sample obtained from the patient with a myeloid malignancy;
(ii) comparing the level of differentiated monocytic AML in (i) with a predetermined cut-off value for differentiated monocytic AML,
   wherein if the level of differentiated monocytic AML determined in the patient sample is not higher than the predetermined cut-off value, the patient is selected for treatment with the anti-CD70 antibody or CD70-binding fragment thereof.

In another aspect, there is provided a method of identifying a patient to be treated with an anti-CD70 antibody or antigen-binding fragment thereof, wherein the patient has a myeloid malignancy and is selected according to a method which comprises the step of:
(i) determining the level of differentiated monocytic AML in a sample obtained from the patient with a myeloid malignancy;
(ii) comparing the level of differentiated monocytic AML in (i) with a predetermined cut-off value for differentiated monocytic AML,
   wherein if the level of differentiated monocytic AML determined in the patient sample is lower than the predetermined cut-off value, the patient is selected for treatment with the anti-CD70 antibody or CD70-binding fragment thereof.

In a further aspect, there is provided a method of identifying a patient to be treated with an anti-CD70 antibody or antigen-binding fragment thereof, wherein the patient has a myeloid malignancy and is selected according to a method which comprises the step of:
(i) determining the level of differentiated monocytic AML in a sample obtained from the patient with a myeloid malignancy;
(ii) comparing the level of differentiated monocytic AML in (i) with a predetermined cut-off value for differentiated monocytic AML,
   wherein if the level of differentiated monocytic AML determined in the patient sample is not lower than the predetermined cut-off value, the patient is selected for treatment with the anti-CD70 antibody or CD70-binding fragment thereof.

In some embodiments, the predetermined cut-off value for differentiated monocytic AML is the average level of differentiated monocytic AML for a control cohort of AML patients. All embodiments described herein relating to the methods of treatment according to the preceding aspects of the invention (see in particular, Section B and Section C) are equally applicable to these further aspects and embodiments of the invention.

### Incorporation by Reference

Various publications are cited in the foregoing description and throughout the following examples, each of which is incorporated by reference herein in its entirety.

### EXAMPLES

### Example 1. Patients With AML with Monocytic Disease Are More Likely to Be Refractory to Venetoclax plus Azacitidine

To test whether differentiation status may predict responsiveness to venetoclax +azacitidine (VEN+AZA) in the clinic, 100 consecutive, newly diagnosed, previously untreated patients with AML who received VEN+AZA were retrospectively reviewed. Several baseline factors were analyzed to determine the ability of each to predict disease that was refractory to treatment as defined by the European Leukemia Network [ELN; lack of complete remission (CR), CR with incomplete recovery of peripheral blood counts (CRi), partial remission (PR), or morphologic leukemia free state (MLFS); Dohner H et al., Blood 2017;129:424-47.]. The median age of the cohort was 72 years; 20 patients (20%) had a documented antecedent hematologic disorder; 64 patients (64%) had adverse risk disease by ELN criteria.

To specifically examine features associated with myeloid differentiation, the FAB (French, American, British) classification system was initially employed. Although this system is no longer employed for clinical purposes, it provides a well-described and clinically associated means to segregate patients with AML by virtue of myeloid differentiation status. In the VEN+AZA-treated patient cohort, 13 patients (13%) were identified as the FAB-M5 subtype, which is defined as a more differentiated phenotype of monocytic AML, 8 (8%) were FAB-M4, and 77 (77%) were FAB-M0 or M1, indicative of a less differentiated phenotype. Univariate analysis revealed sex (P=0.0495), presence of an RAS pathway mutation (P=0.0039), and FAB-M5 maturation state (P<0.0001) to be associated with disease that was refractory to VEN+AZA (Table 2). A multivariate analysis revealed only the FAB-M5 maturation state (P=0.0066) to be predictive of refractory response (Table 2). Specifically, 62% of FAB-M5 patients were refractory to VEN+AZA, whereas 0% of FAB-M4 and only 8% of non-FAB-M5 patients were refractory. In addition, the median overall survival in FAB-M5 patients was 89 days, compared with 518 days for non-FAB-M5 patients (P=0.0039). These findings indicate a strong correlation between myeloid differentiation status and resistance to venetoclax-based therapy.

It should be noted that Kuusanmäki et al. (2020) Haematologica 105(3): 708-720 reported that, based on ex vivo testing, in the total mononuclear cell fraction the highest BCL2/MCLI gene expression ratio was observed in M0/1 and the lowest in M4/5 AML. This group further reported that, based on ex vivo characterization and drug sensitivity testing, the gene expression data of mononuclear cell-enriched AML samples indicated that M4/5 AML have low BCL2 but high MCLI and BCL2A1 expression, consistent with decreased venetoclax sensitivity observed with the total mononuclear cell fraction of M4/5 samples.

TABLE 2
Baseline characteristics and univariate and multivariate logistic
regression analysis of 100 consecutive patients with newly
diagnosed, previously untreated AML who received VEN + AZA

| | | Univariate analysis | | Multivariate analysis | |
|---|---|---|---|---|---|
| | | as a predictor for | | as a predictor for | |
| | | refractory disease | | refractory disease | |
| Baseline | | OR | P | OR | P |
| Variables | Value | (95% CI) | Value | (95% CI) | Value |
| Age | 71.5 | 0.984 | 0.4028 | | |
| (median) | (22-89) | (0.947-1.022) | | | |
| Sex (female) | 51 | 3.401 | 0.0495 | 2.096 | 0.3694 |
| | (51%) | (1.002-11.539) | | (0.417-10.544) | |
| Antecedent | 20 | 0.573 | 0.4884 | | |
| hematologic | (20%) | | | | |
| disorder | | | | | |
| Complex | 28 | 2.667 | 0.0883 | | |
| Cytogenetics | (28%) | (0.863-8.237) | | | |
| ELN | | | | | |
| Prognostic | | | | | |
| Group | | | | | |
| Favorable | 18 | | | | |
| | (18%) | | | | |
| Intermediate | 17 | 4.078 | 0.0697 | | |
| | (17%) | (0.494-33.643) | | | |
| Adverse | 64 | | | | |
| | (64%) | | | | |
| NA | 1 | | | | |
| | (1%) | | | | |
| RAS | 14 | 6.417 | 0.0039 | 2.266 | 0.5080 |
| pathway | (14%) | (1.813-22.708) | | (0.201-25.522) | |
| mutations | | | | | |
| TP53 | 10 | 1.481 | 0.6424 | | |
| | (10%) | (0.282-7.766 | | | |
| IDH1/IDH2 | 27 | NE | 0.9521 | | |
| | 27(%) | | | | |
| NPM1 | 27 | 0.162 | 0.0865 | 0.488 | 0.5967 |
| | (27%) | (0.020-1.298) | | (0.034-6.966 | |
| FLT3-ITD | 18 | 0.663 | 0.6119 | | |
| | (18%) | (0.136-3.273) | | | |
| ASXL1 | 24 | 1.182 | 0.7932 | | |
| | (24%) | (0.339-4.122) | | | |
| FAB | | | | | |
| Classi- | | | | | |
| fication | | | | | |
| M0/M1 | 77 | 0.131 | 0.0008 | | |
| | (77%) | (0.040-0.428) | | | |
| M2 | 1 | | | | |
| | (1%) | | | | |
| M4 | 8 | NE | 0.9745 | | |
| | (8%) | | | | |
| M5 | 13 | 18.285 | <0.0001 | 33.481 | 0.0066 |
| | (13%) | (4.701-71.129) | | (2.657-421.90) | |
| M6a | 1 | | | | |
| | (1%) | | | | |

NA, not available
NE, not estimable

### Example 2. Monocytic AML Is Intrinsically Resistant to VEN+AZA

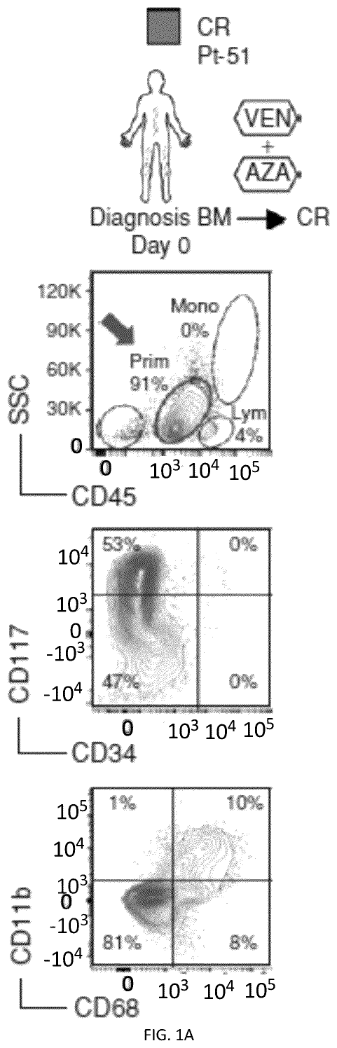

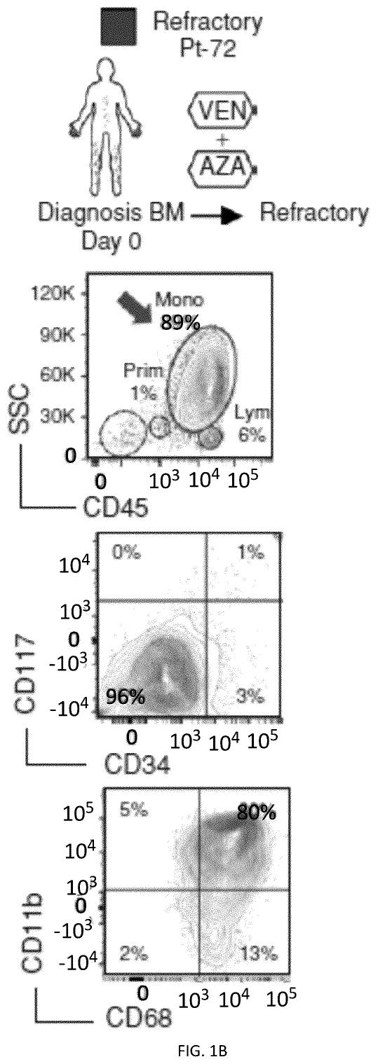

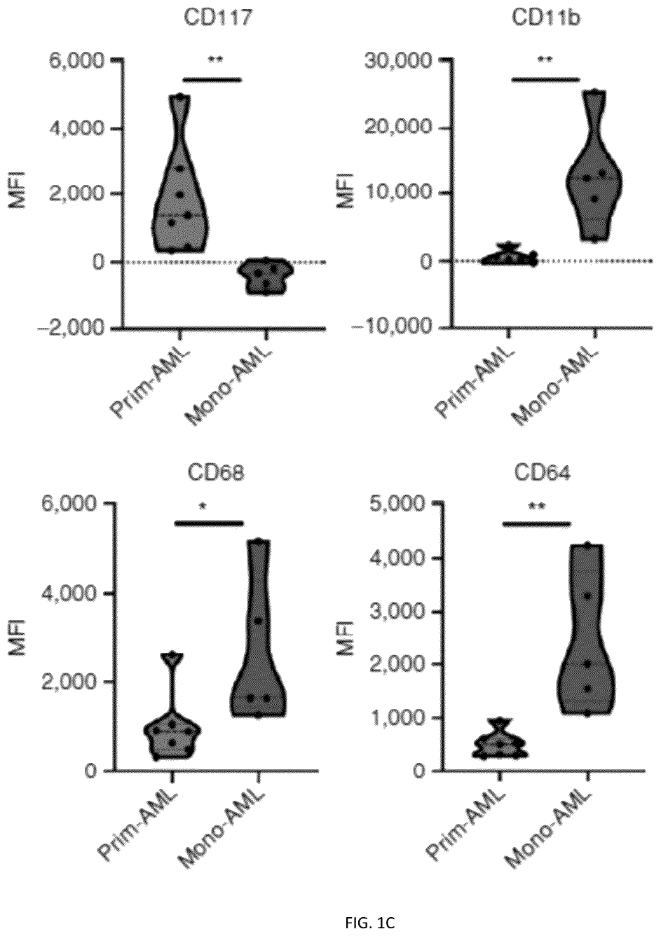

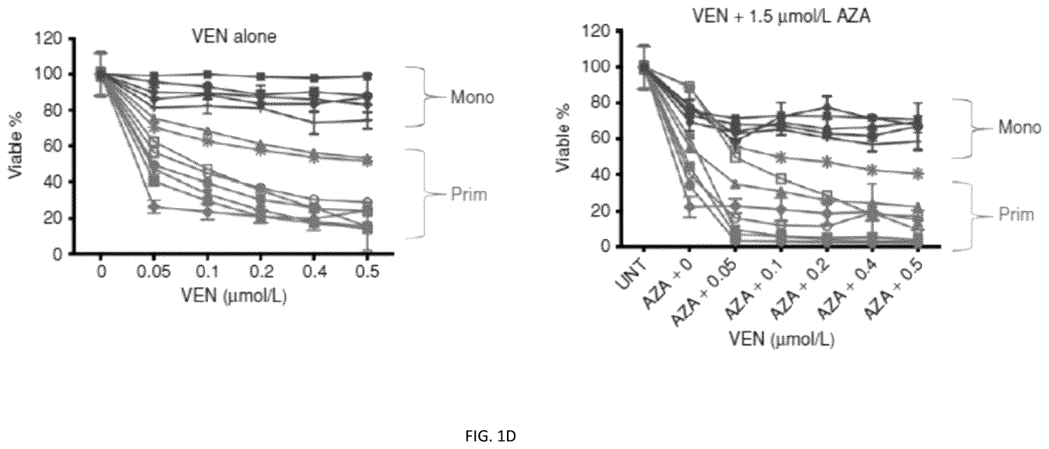

To understand if the lack of response by monocytic AML to VEN+AZA is driven by intrinsic mechanisms, VEN+AZA sensitivity in vitro, where protection from extrinsic factors such as the microenvironment is minimal, was directly evaluated. Because the FAB system is no longer employed for clinical purposes, phenotypic markers were employed that would serve as a surrogate for the FAB-M5 subtype. Previous studies have shown that FAB-M5 patients lose expression of the primitive marker CD117 and upregulate expression of the monocytic markers CD11b, CD68, and CD64. Xu Y et al. (2006) Leukemia 20: 1321-4; Garcia C et al. (2008) Appl Immunohistochem Mol Morphol 16: 417-21; Cascavilla N et al. (1998) Haematologica 83: 392-7; Di Noto R et al. (1996) Br J Haematol 92: 562-4; Naeim F., Atlas of Hematopathology: Morphology, Immunophenotype, Cytogenetics, and Molecular Approaches. 1st ed. London: Academic Press; 2013. pxi, 743 p. Therefore, a multicolor flow cytometry panel including CD117, CD11b, CD68, and CD64 was designed to distinguish patients with monocytic AML (FAB-M5) from patients with primitive AML (FAB-M0/M1/M2). As shown in FIG. 1, this approach readily distinguished two predominant cell populations within patients with AML. For example, patient 51 (Pt-51; a typical FAB-M0/M1/M2) presented with a single dominant disease population that was phenotypically primitive as evidenced by CD45-medium/SSC-low/CD117+/CD11b−/CD68− (FIG. 1A). This patient achieved complete remission (CR) with VEN+AZA treatment. In contrast, Pt-72 (a typical FAB-M5) was refractory to VEN+AZA and presented with dominant monocytic disease that was CD45-bright/SSC-high/CD117−/CD11b+/CD68+ (FIG. 1B). Analysis of an additional 12 primary AML specimens confirmed the phenotypic profile for primitive versus monocytic specimens (FIG. 1C). Hereafter, these AMLs are noted as “prim-AML” or “mono-AML,” respectively.

Multiple studies have suggested that leukemic stem cells (LSC) are an important target of AML therapies. Pollyea DA et al. (2017) Blood 129: 1627-35. Previous studies have shown that a phenotype of low reactive oxygen species (ROS-low) enriches for functionally defined LSCs. Lagadinou ED et al. (2013) Cell Stem Cell 12: 329-41; Pei S et al. (2018) Cell Stem Cell 23: 86-100.e6. Therefore, to more directly assess drug responsiveness in the LSC subpopulation, ROS-low cells were isolated from prim-AML and mono-AML specimens. Because mono-AML has never been directly characterized by ROS level, colony-forming unit (CFU) assays confirmed that the ROS-low phenotype enriches for stem/progenitor potential in mono-AML. These data indicate the ROS-low phenotype strongly enriches for stem/progenitor potential in mono-AML, similar to what was reported for prim-AML. The ROS-low subpopulations from prim-AML or mono-AML were then treated with VEN+AZA in vitro. Results showed that ROS-low LSCs of the mono-AML specimens were significantly more resistant than those of the prim-AML specimens (FIG. 1D), suggesting the refractory responses seen in FAB-M5 patients can be at least partially attributed to intrinsic molecular mechanisms uniquely present in monocytic AML cells. FIG. 1 adapted from Pei et al. (2020).

### Example 3. Monocytic AML Loses Expression of the Venetoclax Target BCL-2

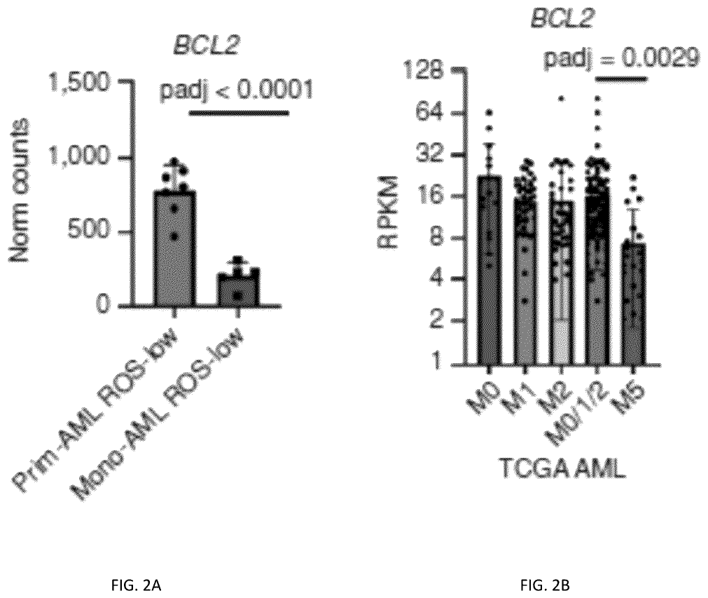

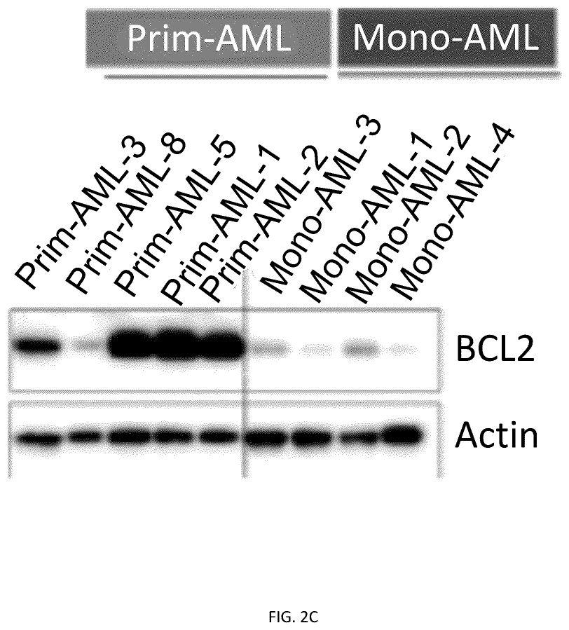

Venetoclax is a BCL-2-specific inhibitor, and several studies have shown that BCL-2 expression strongly correlates with venetoclax sensitivity in vitro. Souers A J et al. (2013) Nat Med 19: 202-8; Pan R et al. (2014) Cancer Discov 4: 362-75. Among genes related to apoptosis regulation, analysis revealed significant and consistent loss of BCL2 in mono-AMLs (N 5), compared with the prim-AMLs (N 7; FIG. 2A). Analysis of the TCGA AML dataset also showed progressive loss of BCL2 gene expression through stages of AML morphologic maturation (FAB-M0 to FAB-M5). As a result, significantly lower expression of BCL2 was observed in FAB-M5 relative to FAB-M0/M1/M2 in the TCGA AML dataset (FIG. 2B). Further, reduced expression of BCL-2 in mono-AML was confirmed at the protein level (FIG. 2C). Interestingly, loss of BCL-2 also occurs during normal monocytic development. Novershtern N et al. (2011) Cell 144: 296-309; Lara-Astiaso D et al. (2014) Science 345: 943-9. Consistent loss of BCL-2 was found at the monocytic stage in both human and murine systems.

Together, these analyses indicate BCL-2 loss is a conserved biological feature during both normal and malignant monocytic development. Further, the data suggest BCL-2 loss in monocytic AML may drive resistance to venetoclax-based therapies. FIG. 2 adapted from Pei et al. (2020).

### Example 4. Venetoclax Plus Azacitidine (VEN+AZA) Selects Monocytic Disease at Relapse

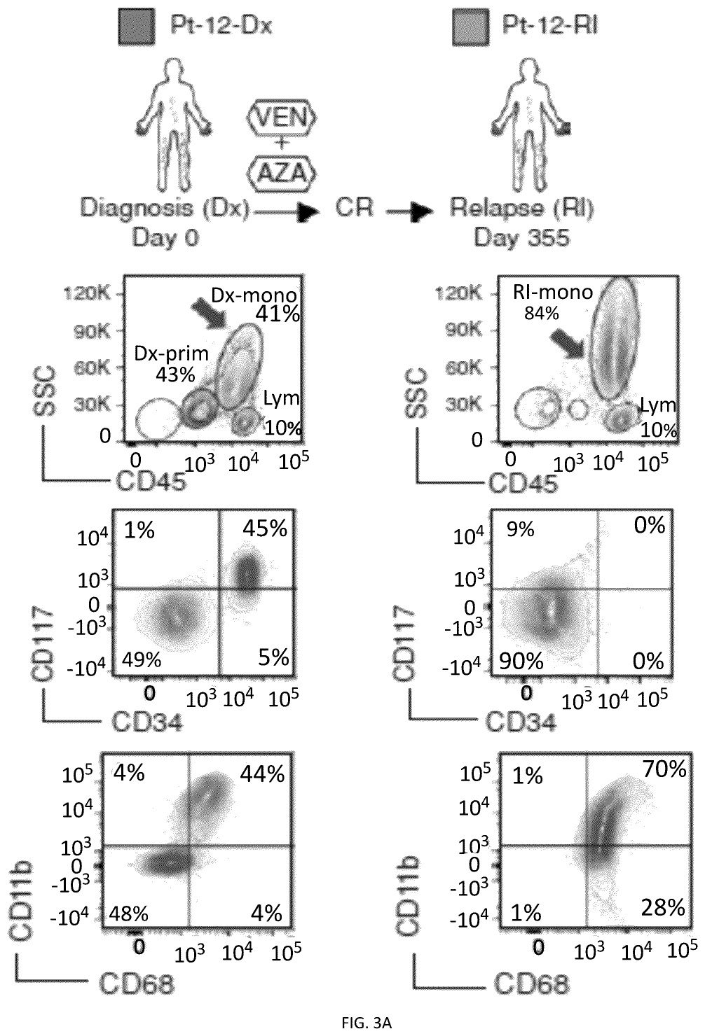

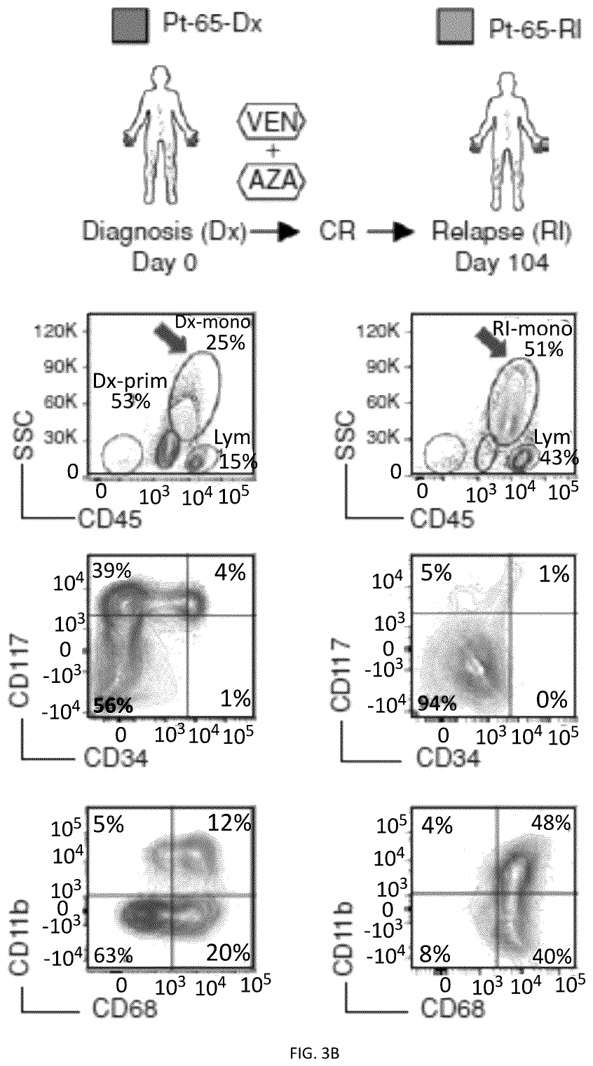

Based on the above findings, the extent to which monocytic disease is evident in patients who initially responded but then relapsed on VEN+AZA therapy was investigated. In analyzing patients with AML prior to VEN+AZA treatment, it was noted that the majority of patients actually present with tumors showing a mixture of the monocytic and primitive phenotype, termed “MMP-AML” (for mixed monocytic/primitive-AML). Characteristics of two patients with MMP-AML (Pt-12, Pt-65) were analyzed during the course of treatment (FIGS. 3A and 3B). Upon relapse after an initial complete remission, both patients showed almost complete loss of the primitive subpopulation and emergence of a dominant monocytic phenotype (CD45-bright/SSC-high/CD117−/CD11b+/CD68+). Thus, VEN+AZA treatment appeared to induce striking in vivo selection for the monocytic subpopulation in each patient (FIGS. 3A and 3B).

Of note, this monocytic selection phenotype seemed to be a unique clinical characteristic of VEN+AZA therapy. Indeed, previous analyses of patients treated with conventional chemotherapy have shown consistent enrichment of more primitive LSC phenotypes. Ho T C et al. (2016) Blood 128: 1671-8.

To further corroborate this finding, RNA-seq data of 11 pairs of diagnostic and relapsed specimens after conventional chemotherapy from a separate study by Shlush and colleagues were analyzed. Shlush L I et al. (2017) Nature 547: 104-8. In this setting, a gain of the LSC gene-expression signature, and loss of monocytic markers (CD11b and CD68) and a monocytic gene-expression signature at relapse was observed, suggesting suppression of the myeloid phenotype following chemotherapy.

Lastly, paired diagnosis versus relapse specimens from 6 patients with AML treated with conventional chemotherapy were compared. In no case was a monocytic phenotype evident at relapse. In fact, for 2 patients with monocytic characteristics at diagnosis, conversion to a more primitive phenotype at relapse was observed.

Together, these data suggest that relapse following conventional chemotherapy strongly favors a primitive phenotype, and that selection of a monocytic phenotype at relapse appears to be a distinct characteristic of VEN+AZA therapy. FIG. 3 adapted from Pei et al. (2020).

### Example 5. Treatment of Patients Having Reduced Sensitivity to Venetoclax—Cusatuzumab Alone

Two or more adult human patients having AML that has a reduced sensitivity or is refractory to venetoclax are selected for study. The patients are administered cusatuzumab intravenously (i.v.) in a dose of about 10 mg/kg once every 12-14 days. The patients' blast counts are measured prior to beginning the cusatuzumab (pre-treatment baseline) and then monitored about weekly for at least the period ending two weeks after the last or most recent dose of cusatuzumab. Flow cytometry is used to determine the proportion of blast cells using the number of CD45^{dim}, SSC^{low} cells relative to total cell number. A reduction in blast counts of at least 5% from pre-treatment baseline indicates successful intervention.

### Example 6. Treatment of Patients Having Reduced Sensitivity to Venetoclax—Cusatuzumab in Combination with Venetoclax

Two or more adult human patients having AML that has a reduced sensitivity or is refractory to venetoclax are selected for study. The patients are administered cusatuzumab intravenously (i.v.) in a dose of about 10 mg/kg once every 12-14 days. Beginning with the second dose of cusatuzumab, the patients are also administered venetoclax orally (p.o.) daily at a dose of 400-600 mg, with a ramp-up dosing schedule beginning with a first dose of 100 mg and increasing by 100 mg/day until reaching the target daily dose of 400-600 mg. The patients' blast counts are measured prior to beginning the cusatuzumab (pre-treatment baseline) and then monitored about weekly for at least the period ending two weeks after the last or most recent dose of cusatuzumab. Flow cytometry is used to determine the proportion of blast cells using the number of CD45^{dim}, SSC^{low} cells relative to total cell number. A reduction in blast counts of at least 5% from pre-treatment baseline indicates successful intervention.

### Example 7. Treatment of Patients Having Reduced Sensitivity to Venetoclax—Cusatuzumab in Combination with Venetoclax and Azacitidine

Two or more adult human patients having AML that has a reduced sensitivity or is refractory to venetoclax are selected for study. The patients are administered cusatuzumab intravenously (i.v.) in a dose of about 10 mg/kg once every 12-14 days. Beginning with the second dose of cusatuzumab, the patients are also administered venetoclax orally (p.o.) daily at a dose of 400-600 mg, with a ramp-up dosing schedule beginning with a first dose of 100 mg and increasing by 100 mg/day until reaching the target daily dose of 400-600 mg. Also beginning with the second dose of cusatuzumab, the patients are also administered azacitidine 75 mg/m² subcutaneously (s.c.) or i.v. daily for 7 days; a repeat cycle is administered once every 4 weeks. The patients' blast counts are measured prior to beginning the cusatuzumab (pre-treatment baseline) and then monitored about weekly for at least the period ending two weeks after the last or most recent dose of cusatuzumab. Flow cytometry is used to determine the proportion of blast cells using the number of CD45^{dim}, SSC^{low} cells relative to total cell number. A reduction in blast counts of at least 5% from pre-treatment baseline indicates successful intervention.

### Example 8. Monocytic AML Cells Express Significantly Higher CD70 Levels Compared to Less Differentiated Primitive AML Cells

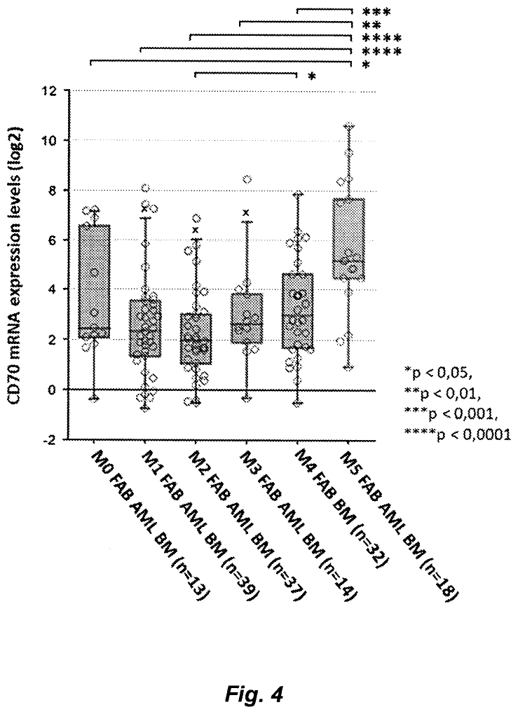

An analysis of CD70 mRNA expression showed on average at least 6 times higher CD70 expression on the transcriptional level in bone marrow samples from AML patients with FAB M5 subtype (FIG. 4), containing at least 80% of cells differentiated in a direction of monocytic cells (monocytic AML). Monocytic AML cells are phenotypically different from less differentiated AML cells (primitive AML and AML with maturation, FAB MO-M2) and classified as CD45^{bright}/SSC^{high}/CD117⁻/CD11b⁺/CD68⁻. This is in contrast to primitive AML cells, which show CD45^{medium}/SSC^{low}/CD117⁺/CD11b⁻/CD68⁻ phenotype in flow cytometry analysis (Pei et al. 2020). An analysis of responses to VEN+AZA combination in FAB AML subtypes showed monocytic blasts from FAB M5 subtype to be associated with a disease refractory to the VEN+AZA combination. Specifically, 62% of FAB M5 and only 8% of non-FAB M5 patients were refractory to VEN+AZA combination (Pei et al 2020). Interestingly, myelomonocytic FAB M4 subtype also has increased levels of CD70 when compared to less differentiated subtypes. Other authors also described this subtype to be associated with resistance to VEN+AZA (Zhang et al, 2020, Kuusanmäki et al 2017). FAB M4 subtype is a mixed phenotype leukemia, since it consists of a combination of clones with different stages of myeloid differentiation and at least 20% of monocytic blasts. VEN+AZA drug combination shows better efficacy in this subgroup, but monocytic AML cells present in this subgroup may also potentially increase the risk of an early relapse (Zhang et al. 2020). Moreover, both M4 and M5 subtypes have the lowest BCL2/MCL1 gene expression ratio, which is associated with resistance to Bc1-2 inhibition (Kuusanmäki et al 2020).

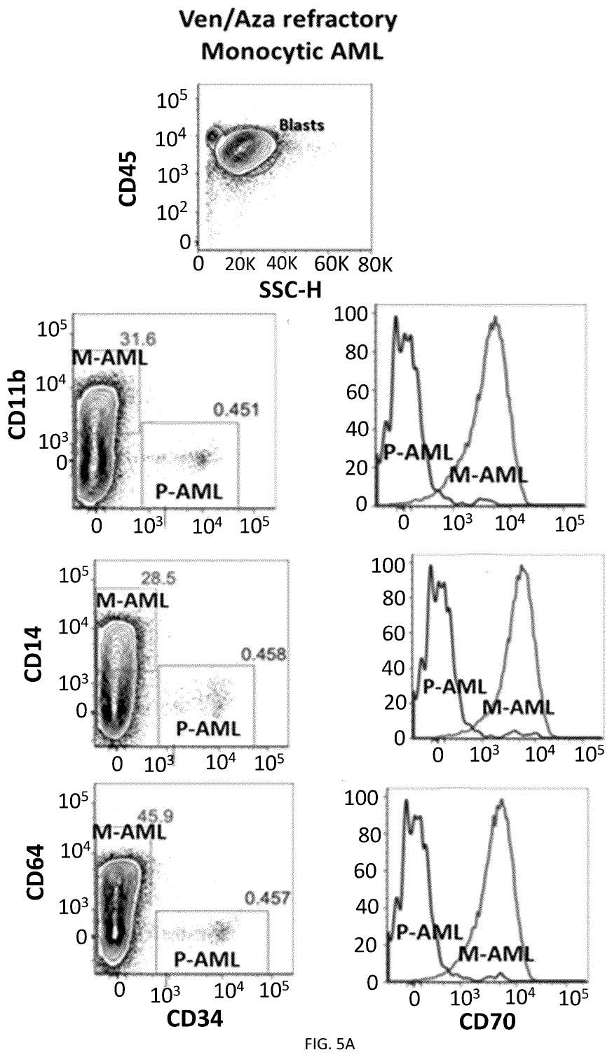

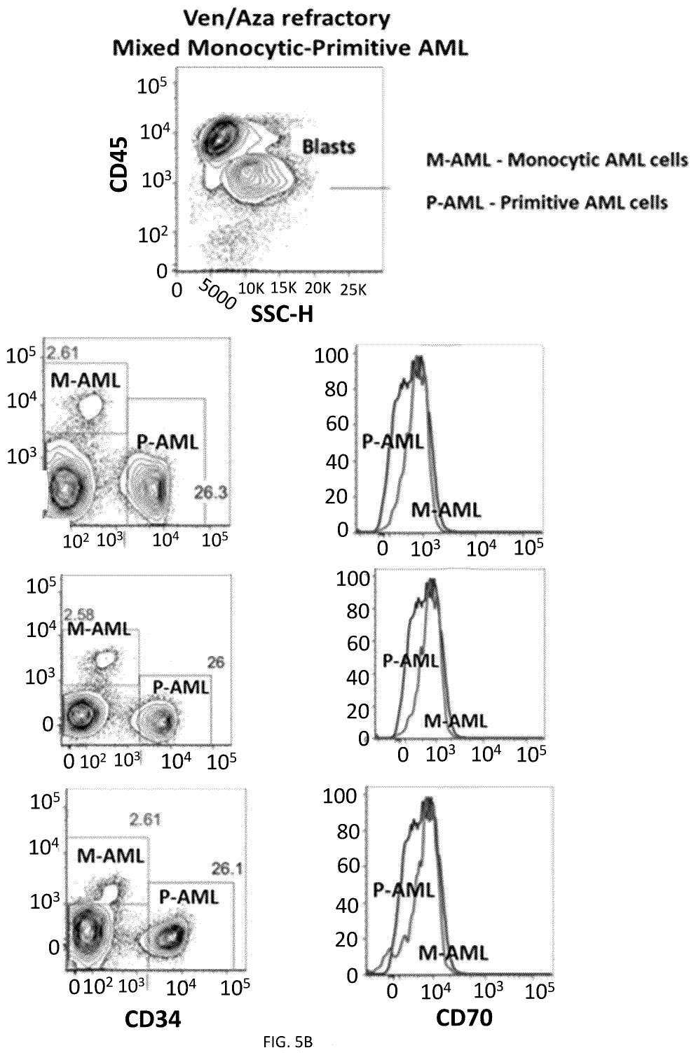

Bone marrow samples from patients with monocytic and mixed phenotype AML were tested for CD70 expression and the phenotype of CD70 positive cells was confirmed by flow cytometry (FIG. 5). Cytometric analysis confirmed that a high CD70 expression on the plasma membrane of malignant cells was present on VEN+AZA resistant monocytic AML cells with CD45^{bright}/SSC^{high}/CD34⁻/CD117⁻/CD11b⁻/CD68⁺/CD14⁺/CD64⁺ phenotype (FIG. 5). Typically, monocytic disease samples showed the highest CD70 expression (FIG. 5A), whereas primitive blasts showed only very limited CD70 expression. Occasionally mixed phenotype samples consisting of monocytic and primitive leukemic cells showed a relatively high CD70 expression on both monocytic and primitive AML cells, but generally primitive cells showed low CD70 expression on the cell surface. An example of mixed phenotype sample with a high CD70 expression on primitive and monocytic AML cells coming from VEN+AZA refractory patient is presented in FIG. 5B.

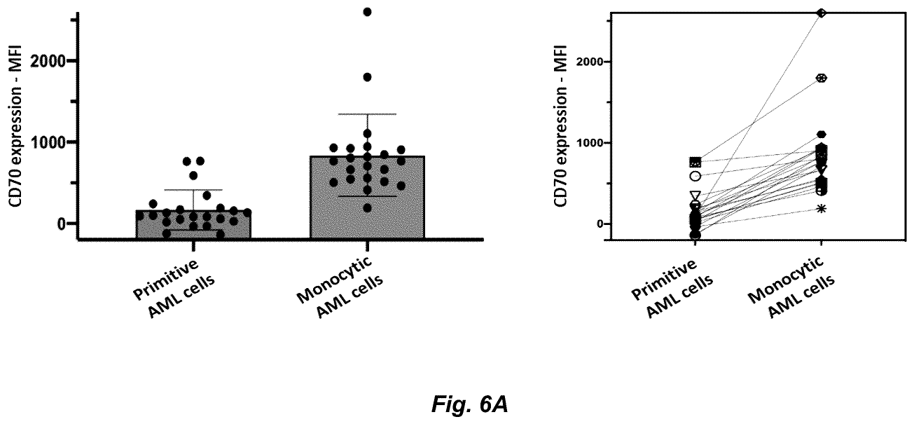

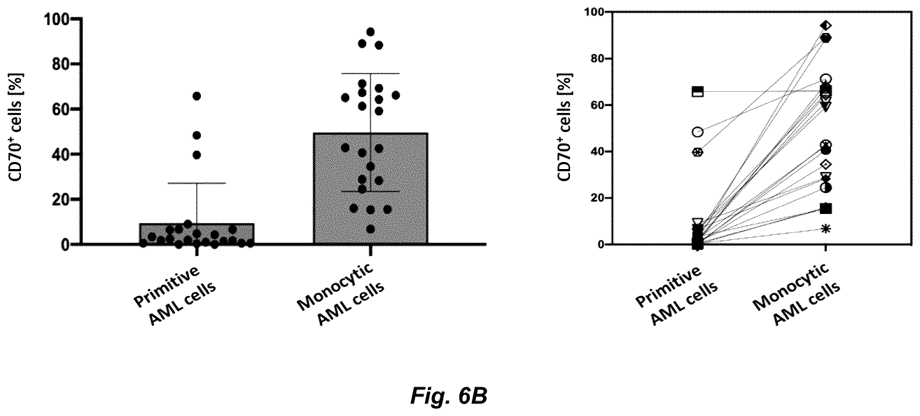

Because AML samples often show mixed phenotype with different ratio of primitive and monocytic malignant cells, a comparison of CD70 expression on monocytic and primitive cell subpopulations was performed. Flow cytometry analysis showed about 6 times higher median fluorescence intensity for CD70 in the case of monocytic AML cells in comparison to primitive AML cells. This confirmed a higher expression of CD70 on monocytic AML cells (FIG. 6A left) and was comparable to the data obtained from CD70 mRNA analysis (FIG. 4). A paired analysis per sample also showed that CD70 expression level is higher on monocytic AML cells than on primitive AML cells present in the same patient sample (FIG. 6A right). Only a limited number of samples showed an equally high CD70 expression on primitive and monocytic cells. Calculation of the percentage of CD70 positive cells also confirmed the data from the protein expression level. On average over 50% of monocytic AML cells present in a sample showed a high CD70 expression, whereas less than 10% of primitive AML cells showed CD70 expression (FIG. 6B left). Moreover, 95% of samples had a higher percentage of CD70 positive cells on monocytic AML cells than the average for primitive AML cells. A paired analysis also confirmed the fact that a higher percentage of CD70 positive monocytic malignant cells than primitive AML cells was present in the same sample (FIG. 6B right).

### Example 9. CD70 Positive VEN+AZA Resistant Monocytic AML Cells are Efficiently Killed by Cusatuzumab-Mediated ADCC

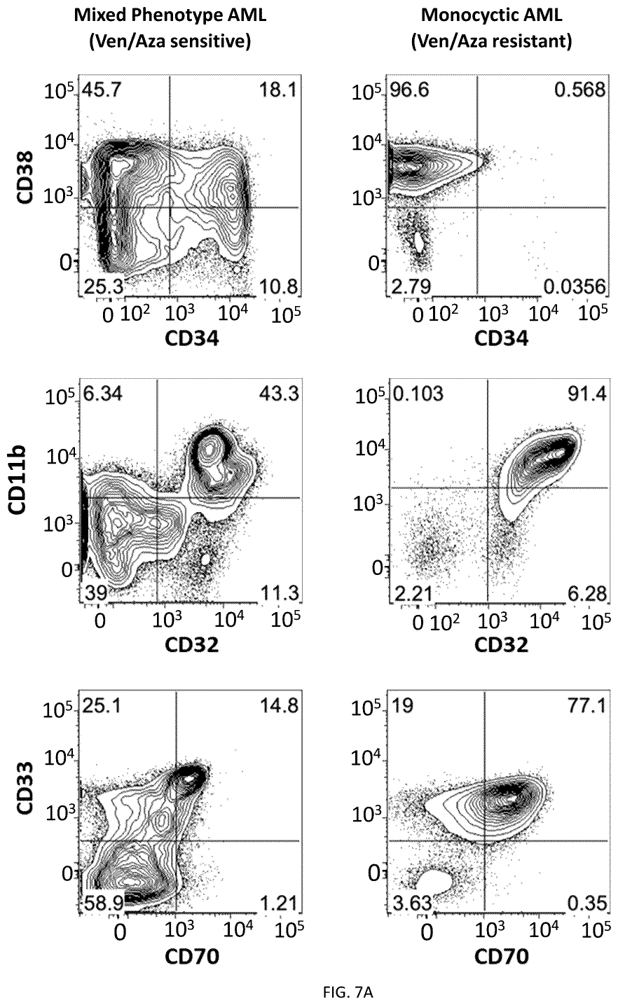

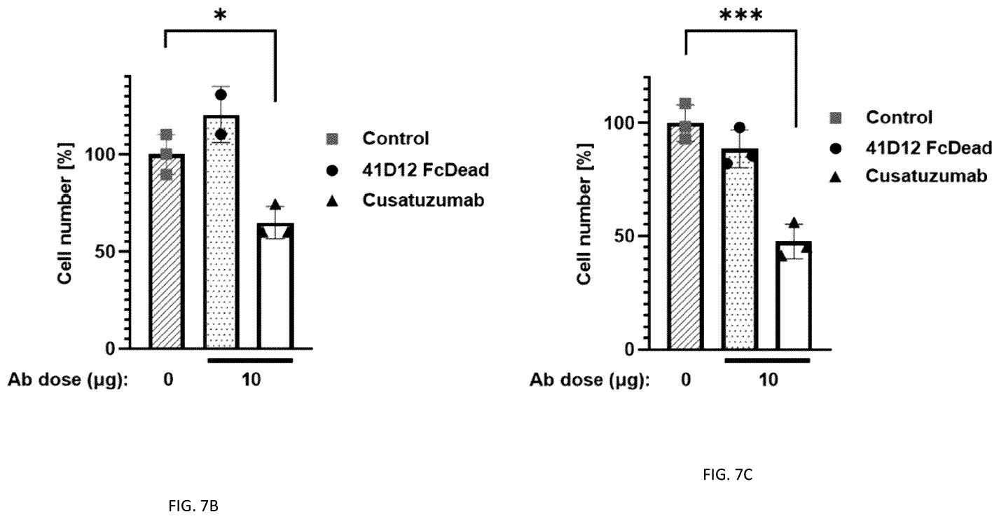

Cusatuzumab is an afucosylated anti-human CD70 antibody with enhanced properties to mediate NK-dependent antibody-dependent cellular cytotoxicity (ADCC) (Silence et al. 2014). VEN+AZA resistant CD70 positive monocytic AML (CD45^{bright}/SSC^{high}/CD38⁺/CD34⁻/CD33⁺/CD11b⁺/CD70⁺) and mixed phenotype samples containing CD70 positive monocytic cells (CD45^{bright}/SSC^{high}/CD38⁺/CD34⁻/CD33⁺/CD11b⁺/CD70⁺) and CD70 negative VEN+AZA sensitive primitive cells (CD45^{medium}/SSC^{low}/CD34⁻/CD33⁺/CD11b⁺/CD70⁻ containing both CD38⁺ and CD38⁻ populations) (FIG. 7A) were tested for sensitivity to cusatuzumab-mediated ADCC. Both types of primary bone marrow samples were treated with cusatuzumab at 10 μg/ml concentration and co-incubated with human NK cells isolated by a negative selection from healthy donor PBMCs. NK cells were added in 1:5 and 1:15 target to effector cells (T:E) ratio to monocytic AML and mixed phenotype bone marrow samples, respectively. Cells were co-cultured for 24 hours at 37° C. in cell culture incubator. Flow cytometry analysis was performed in order to measure number of primitive and monocytic AML cells and estimate the level of ADCC for particular sample. Monocytic cells in both samples were significantly targeted by cusatuzumab-mediated NK cell-dependent ADCC (FIG. 7B and 7C, respectively). Blocking anti-CD70 41D12 FcDead antibody with reduced effector functions was used as a negative control and no significant antibody-specific effect in targeting CD70 positive monocytic cells was detected for the blocking antibody (FIG. 7B and 7C). This supports the specificity of cusatuzumab-mediated effects in the targeting of CD70-positive VEN+AZA resistant monocytic AML cells.

### Example 10. Cusatuzumab Effectively Targets CD70 Positive LSCs from VEN+AZA Resistant Monocytic AML Samples

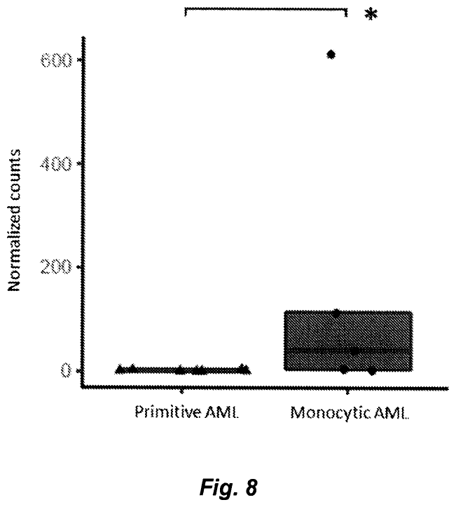

ROS-low enriched leukemic stem cells (LSCs) from primitive and monocytic AML differ significantly in their properties, since ROS-low LSCs from monocytic AML are less dependent on BCL2 protein for their survival and show increased resistance to Venetoclax (Pei et al. 2020). Transcriptomic analysis of samples from primitive and monocytic AML and, in particular, comparison of CD70 expression in the subpopulation enriched for ROS-low LSCs showed, that expression levels of CD70 in ROS-low LSCs from monocytic disease are significantly higher when compared to those in ROS-low LSCs from primitive AML samples (FIG. 8).

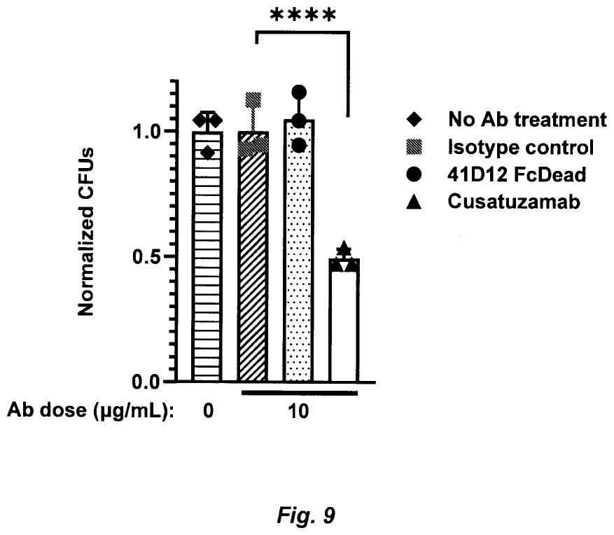

In order to test if CD70 positive LSCs from VEN+AZA resistant monocytic AML bone marrow samples could be targeted by cusatuzumab, bone marrow samples coming from VEN+AZA resistant samples were first incubated with cusatuzumab (10 μg/ml) and NK cells isolated from PBMCs from healthy donor (1:5 T:E ratio). After a 24-hour incubation samples were moved to methocult medium and further cultured in order to check if LSCs were targeted by cusatuzumab-mediated ADCC. No treatment, human IgG1 isotype control and blocking 41D12 FcDead antibody were used as negative controls. Cells were incubated in cell culture incubator at 37° C. for 14 days and colony forming units (CFU) were estimated by counting growing colonies. In the conditions where cusatuzumab was used, a clear drop in the number of growing colonies was observed in comparison to the control with no treatment (FIG. 9). No significant effect of the isotype control nor the blocking anti-CD70 antibody was detected.

### Example 11. Cusatuzumab Significantly Reduces CD70 Positive VEN+AZA Resistant Monocytic AML Cells in Patient-Derived Xenograft Mouse Model via an NK-Dependent Mechanism

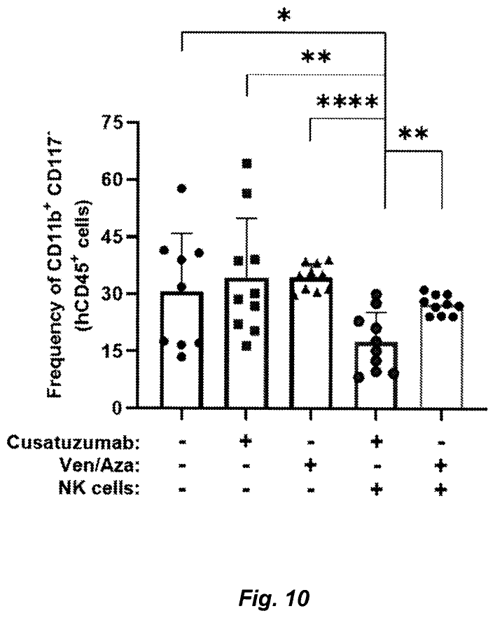

Patient samples injected in NSGS mice engraft in mouse bone marrow. The efficiency of anti-leukemic compounds can be measured stringently using therapeutic approaches after full engraftment of patient-derived samples and by determination of the reduction of malignant cells in mouse bone marrow. 2×10⁶ cells from bone marrow from VEN+AZA resistant monocytic AML per NSGS mouse were engrafted for 42 days. One cohort of animals was treated with vehicle, combination of 100 mg/kg Ven and 3 mg/kg Aza or 10 mg/kg cusatuzumab. Second cohort was first infused with 1.5×10⁶ NK cells isolated from PBMCs from healthy donor and then treated with the same drug combinations as the first cohort. Animals were treated every 3 days with vehicle, VEN+AZA combination or cusatuzumab. One day after the third dose animals were sacrificed and bone marrow from femur was isolated and samples were analysed by flow cytometry to determine the number of monocytic AML cells. Flow cytometry analysis showed a significant reduction in malignant human CD45⁺CD11b⁺CD117⁻ cells in animals treated with cusatuzumab in the presence of human NK cells, but no significant effect was observed for VEN+AZA nor cusatuzumab in the absence of human NK cells nor for VEN+AZA in the presence of NK cells (FIG. 10). Therefore, cusatuzumab is effective in depleting VEN+AZA resistant CD70 positive monocytic AML cells via NK-dependent ADCC in vivo in NSGS mice.

### References

Silence et al. 2014, ARGX-110, a highly potent antibody targeting CD70, eliminates tumors via both enhanced ADCC and immune checkpoint blockade, MAbs, March-April 2014;6(2):523-32
Pei et al. 2020, Monocytic Subclones Confer Resistance to Venetoclax-Based Therapy in Patients with Acute Myeloid Leukemia, Cancer Discov 2020; 10:536-51
Zhang et al. 2020, Integrated analysis of patient samples identifies biomarkers for venetoclax efficacy and combination strategies in acute myeloid leukemia, Nature Cancer, Vol 1 August 2020: 826-839
Kuusanmäki et al 2020, Phenotype-based drug screening reveals association between venetoclax response and differentiation stage in acute myeloid leukemia, Haematologica 2020, Vol 105(3):708-720
Kuusanmäki et al 2017, Single-Cell Drug Profiling Reveals Maturation Stage-Dependent Drug Responses in AML, Blood (2017) 130 (Supplement 1): 3821

1-50. (canceled)

51. A method of treating a myeloid malignancy in a human subject resistant to BCL-2 inhibitor treatment, comprising administering to the subject a therapeutically effective amount of an antibody or antigen binding fragment thereof that binds to CD70.

52. The method of claim 51, wherein:
(a) the BCL-2 inhibitor is venetoclax or a pharmaceutically acceptable salt thereof; and/or
(b) the myeloid malignancy is selected from the group consisting of acute myeloid leukemia (AML), myelodysplastic syndromes (MDS), myeloproliferative neoplasms (MPN), chronic myeloid leukemia (CML), and chronic myelomonocytic leukemia (CMML).

53. The method of claim 52, wherein the AML is monocytic AML, optionally wherein the AML is differentiated monocytic AML.

54. The method of claim 53, wherein the human subject is identified as having differentiated monocytic AML on the basis of differential expression level(s) of at least one marker of malignant myeloid cells selected from the group consisting of: BCL-2, CD117, CD11b, CD68, CD64, BCL2A1, and MCL1.

55. The method of claim 54, wherein the expression level(s) of at least one of BCL-2 and CD117 is downregulated, and the expression level(s) of at least one of CD11b, CD68, CD64, CD70, BCL2A1, and MCL1 is upregulated.

56. The method of claim 51, wherein CD70 expression level is upregulated compared to the CD70 expression level as measured before or during the BCL-2 inhibitor treatment.

57. The method of claim 51, wherein the human subject has a clinical history comprising:
(i) treatment with a BCL-2 inhibitor, and absence of a remission in response to the treatment with the BCL-2 inhibitor; or
(ii) treatment with a BCL-2 inhibitor, partial or complete remission, and partial or complete relapse;
optionally wherein the treatment with the BCL-2 inhibitor further comprised treatment with a hypomethylating agent (HMA).

58. The method of claim 51, further comprising:
(i) a hypomethylating agent (HMA) co-administered with the antibody or antigen binding fragment thereof that binds to CD70, optionally wherein the HMA is selected from the group consisting of azacitidine, decitabine, and guadecitabine; or
(ii) a BCL-2 inhibitor co-administered with the antibody or antigen binding fragment thereof that binds to CD70, optionally wherein the BCL-2 inhibitor is venetoclax or a pharmaceutically acceptable salt thereof.

59. The method of claim 51, wherein the antibody or antibody binding fragment that binds to CD70 comprises:
(i) a variable heavy chain domain (VH) comprising an HCDR1, HCDR2, and HCDR3, and a variable light chain domain (VL) comprising an LCDR1, LCDR2, and LCDR3, wherein HCDR1, HCDR2, HCDR3, LCDR1, LCDR2, and LCDR3 comprise the amino acid sequences of SEQ ID NOs: 1, 2, 3, 4, 5, and 6, respectively;
(ii) a VH comprising an amino acid sequence at least 90% identical to SEQ ID NO: 7 and a VL comprising an amino acid sequence at least 90% identical to SEQ ID NO: 8; and/or
(iii) a VH comprising an amino acid sequence identical to SEQ ID NO: 7 and a VL comprising an amino acid sequence identical to SEQ ID NO: 8.

60. The method of claim 51, wherein the antibody that binds to CD70 is cusatuzumab.

61. A method of treating a myeloid malignancy in a human subject, said method comprising the steps of:
(a) selecting a subject having a myeloid malignancy that has reduced sensitivity to or is refractory to a BCL-2 inhibitor, optionally wherein the BCL-2 inhibitor is venetoclax or a pharmaceutically acceptable salt thereof; and
(b) administering to the subject an antibody or antigen binding fragment thereof that binds to CD70.

62. The method of claim 61, wherein the myeloid malignancy is selected from the group consisting of acute myeloid leukemia (AML), myelodysplastic syndromes (MDS), myeloproliferative neoplasms (MPN), chronic myeloid leukemia (CML), and chronic myelomonocytic leukemia (CMML), optionally wherein the AML is monocytic AML.

63. The method of claim 61, wherein step (a) comprises determining an expression level of:
(i) at least one marker of malignant myeloid cells selected from the group consisting of:
BCL-2, CD117, CD11b, CD68, CD64, BCL2A1; and/or (ii) CD70.

64. The method of claim 63, wherein:
(i) at least one of BCL-2 and CD117 is downregulated, and at least one of CD11b, CD68, CD64, CD70, BCL2A1, and MCL1 is upregulated; or
(ii) CD70 is upregulated compared to a CD70 expression level as measured before or during a BCL-2 inhibitor treatment.

65. The method of claim 61, wherein the human subject has a clinical history comprising:
(i) treatment with a BCL-2 inhibitor, and absence of a remission in response to the treatment with the BCL-2 inhibitor; or
(ii) treatment with a BCL-2 inhibitor, partial or complete remission, and partial or complete relapse;
optionally wherein the treatment with the BCL-2 inhibitor further comprised treatment with a hypomethylating agent (HMA).

66. The method of claim 61, further comprising:
(i) a hypomethylating agent (HMA) co-administered with the antibody or antigen binding fragment thereof that binds to CD70, optionally wherein the HMA is selected from the group consisting of azacitidine, decitabine, and guadecitabine; or
(ii) a BCL-2 inhibitor co-administered with the antibody or antigen binding fragment thereof that binds to CD70, optionally wherein the BCL-2 inhibitor is venetoclax or a pharmaceutically acceptable salt thereof.

67. The method of claim 61, wherein the antibody or antibody binding fragment that binds to CD70 comprises:
(i) a variable heavy chain domain (VH) comprising an HCDR1, HCDR2, and HCDR3, and a variable light chain domain (VL) comprising an LCDR1, LCDR2, and LCDR3, wherein HCDR1, HCDR2, HCDR3, LCDR1, LCDR2, and LCDR3 comprise the amino acid sequences of SEQ ID NOs: 1, 2, 3, 4, 5, and 6, respectively;
(ii) a VH comprising an amino acid sequence at least 90% identical to SEQ ID NO: 7 and a VL comprising an amino acid sequence at least 90% identical to SEQ ID NO: 8; and/or
(iii) a VH comprising an amino acid sequence identical to SEQ ID NO: 7 and a VL comprising an amino acid sequence identical to SEQ ID NO: 8.

68. The method of claim 61, wherein the antibody that binds to CD70 is cusatuzumab.

69. A method of identifying and treating a patient with a myeloid malignancy, the method comprising the steps of:
(a) measuring the myeloid differentiation status of the patient;
(b) determining whether the patient has differentiated monocytic AML; and
(iii) administering an anti-CD70 antibody or CD70-binding fragment thereof to the patient identified as a patient having differentiated monocytic AML.

70. The method of claim 69, wherein a bone marrow sample of the patient is determined to comprise CD45^{bright}/SSC^{high}/CD38⁺/CD34⁺/CD33⁺/CD11b⁺/CD70⁺ phenotype cells or CD45^{bright}/SSC^{high}/CD34⁺/CD117⁻/CD11b⁺/CD68⁺/CD14⁺/CD64⁺ phenotype cells.
